(19) United States
(12) Patent Application Publication
(10) Pub. No.: US 2022/0073932 A1
(43) Pub. Date: Mar. 10, 2022

(54) ANTISENSE OLIGONUCLEOTIDES FOR ALLELE SPECIFICITY

(71) Applicant: ARNAY Sciences LLC, Shrewsbury, MA (US)

(72) Inventor: Sudhir Agrawal, Shrewsbury, MA (US)

(21) Appl. No.: 17/478,293

(22) Filed: Sep. 17, 2021

Related U.S. Application Data
(63) Continuation of application No. PCT/US20/23592, filed on Mar. 19, 2020.
(60) Provisional application No. 62/821,722, filed on Mar. 21, 2019.

(51) Int. Cl. C12N 15/113; A61K 31/7088

(52) U.S. Cl. CPC C12N 15/1137; A61K 31/7088; C12N 2310/314; C12N 2310/315; C12N 2310/11

(57) ABSTRACT

The present invention provides an antisense oligonucleotide compound 17 to 25 nucleotides in length comprising at least 12 contiguous nucleobases complementary to an equal length portion of a target RNA sequence, wherein the antisense oligonucleotide compound comprises a 3′ domain and a 5′ domain, wherein the 3′ domain is 10 to 12 nucleotides in length and each nucleotide comprises a deoxyribonucleotide and a phosphodiester or phosphothioate internucleotide linkage or combinations thereof, and wherein the 5′ domain is 5 to 15 nucleotides in length, and wherein the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone. The invention further provides methods of using the antisense oligonucleotide compounds as described herein.

### RELATED APPLICATIONS

This application is a continuation of International Application No. PCT/US20/23592, which designated the United States and was filed on Mar. 19, 2020, published in English, which claims the benefit of U.S. Provisional Application No. 62/821,722, filed on Mar. 21, 2019. The entire teachings of the above applications are incorporated herein by reference.

### BACKGROUND

The potential for the development of an antisense oligonucleotide therapeutic approach was first suggested in articles published 1978. Zamecnik and Stephenson, Proc. Natl. Acad. Sci. U.S.A. 75: 280-284 and 285-288 (1978); discloses that a 13-mer synthetic oligonucleotide that is complementary to a part of the Rous sarcoma virus (RSV) genome inhibits RSV replication in infected chicken fibroblasts and inhibits RSV-mediated transformation of primary chick fibroblasts into malignant sarcoma cells.

An antisense oligonucleotide approach makes use of sequence-specific binding of DNA and/or RNA based oligonucleotides to selected mRNA, microRNA, preRNA or mitochondrial RNA targets and the inhibition of translation that results therefrom. This oligonucleotide-based inhibition of translation and ultimately gene expression is the result of one or more cellular mechanisms, which may include but is not limited to (i) direct (steric) blockage of translation, (ii) RNase H-mediated inhibition, and (iii) RNAi-mediated inhibition (e.g., short interfering-RNA (siRNA), microRNA (miRNA), Modulation of Splicing, Inhibition of noncoding RNA and single-stranded RNAi (ssRNAi)).

The history of antisense technology has revealed that while determination of antisense oligonucleotides that bind to mRNA is relatively straight forward, the optimization of antisense oligonucleotides that have true potential to inhibit gene expression and therefore be good clinical candidates is not. Being based on oligonucleotides, antisense technology has the inherent problem of being unstable in vivo and having the potential to produce off-target effects, for example unintended immune stimulation (Agrawal & Kandimalla (2004) Nature Biotech. 22:1533-1537); https://pubs.rsc.org/en/content/chapter/bk9781788012096-00001/978-1-78801-209-6.

Approaches to optimizing each of these technologies have focused on addressing biostability, affinity to RNA target, cell permeability, and in vivo activity. Often, these have represented competing considerations. For example, early studies were carried out using antisense oligonucleotides containing phosphodiester inter-nucleotide linkages, which proved to be too biologically unstable to be effective in vivo. Thus, there was a focus on modifying antisense oligonucleotides to render them more biologically stable.

Early approaches focused on modifying the inter-nucleotide linkages to make them more resistant to degradation by cellular nucleases. Initial studies were carried out using phosphorothioate, methylphosphonate and phosphoramidates modifications. These studies showed that phosphorothioate antisense had more potent activity than the other two modifications. Increased potency observed with antisense phosphorothioate analogs was primarily due to activation of RNase H, which excises the target RNA at the duplex site. The first in vivo studies of antisense phosphorothioate analogs showed very broad tissue disposition and stability of the administered compound. However, phosphorothioate modification of oligodeoxynucleotides produced unwanted biological activities, including complement activation and immune activation.

Additionally, studies showed that phosphorothioate analogs are susceptible in vivo to degradation by exonucleases, with the primary degradation occurring from the 3′-end of the molecule. As such, approaches to avoid this exonuclease activity have been utilized. One such example is antisense design in which two antisense phosphorothioate oligonucleotides have been linked via their 3′-ends. (Chaix et al., (1996) Biorg. & Med. Chem Letters. V.6(7): 827-832).

To overcome limitations of antisense phosphorothioate oligodeoxynucleotides, various other modifications have been studied. Studies with antisense phosphorothioate oligoribonucleotides and 2′-modified oligoribonucleotides showed that these analogs have less potent antisense activity than phosphorothioate oligodeoxynucleotides, but show desirable characteristics including high affinity and nucleolytic stability and reduced complement activation, but did not activate RNase H. This led to create an antisense in which the desirable characteristics of phosphorothioate oligodeoxy- and oligoribo- or 2′-modified oligoribonucleotides were combined by using the segments of each at defined positions (see e.g., U.S. Pat. Nos. 5,652,355 and 5,652,326). This class of antisense is referred to as hybrid or gapmers and are widely employed in clinical development of antisense drug candidates. While this class of antisense has shown promising results, there have been reports of off target effects due to high affinity and increased nucleolytic stability.

Despite considerable research, the efforts to improve the stability and maintain RNA target recognition, without off-target effects has not generally produced oligonucleotides that would be perceived having higher probability of clinical success. Accordingly, if an oligonucleotide-based approach to down-regulating gene expression is to be successful, there is still a need for optimized antisense oligonucleotides that most efficiently achieve this result. New approaches are therefore needed to mitigate off target activity mediated by RNase H; allow for allele specific knockdown, and allow degradation and mitigate tissue accumulation.

### SUMMARY OF THE INVENTION

The invention provides an antisense oligonucleotide compound 17 to 25 nucleotides in length comprising at least 12 contiguous nucleobases complementary to an equal length portion of a target RNA sequence, wherein the antisense oligonucleotide compound comprises a 3′ domain and a 5′ domain, which is contiguous with the 3′ domain, wherein the 3′ domain is 10 to 12 nucleotides in length and each nucleotide comprises a deoxyribonucleotide and a phosphodiester or phosphothioate internucleotide linkage or combinations thereof; and wherein the 5′ domain is 5 to 15 nucleotides in length, and wherein the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone.

The invention also provides a pharmaceutical composition comprising the antisense oligonucleotide as described herein and a pharmaceutically acceptable carrier.

The invention also provides a method for inhibiting gene expression comprising administering an antisense oligonucleotide as described herein or a composition as described herein, wherein the antisense oligonucleotide is complementary to a nucleotide sequence of a target RNA.

The invention also provides a method for inhibiting allele-specific gene expression comprising administering an antisense oligonucleotide as described herein or a composition as described herein, wherein the antisense oligonucleotide is complementary to a nucleotide sequence of a target allele RNA.

Any of the methods as described herein, can be useful for treating a subject having disease or disorder wherein inhibiting expression of a gene would be beneficial.

### BRIEF DESCRIPTION OF THE DRAWING

FIG. 1 is a schematic of an embodiment of the present invention.

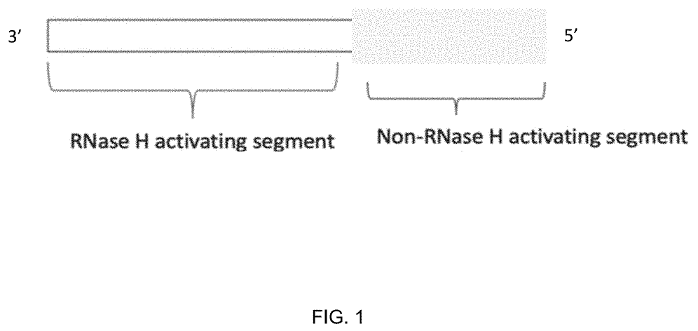

### DETAILED DESCRIPTION

The present invention is directed to compounds, compositions, and methods useful for modulating gene expression using oligonucleotide-based compounds. The compounds of the invention are capable of selectively modulating the expression of an RNA comprising a target sequence. The target RNA could be pre-mRNA, mRNA, noncoding RNA or microRNA.

By convention, sequences discussed herein are set forth 5′ to 3′ unless other specified. Moreover, a strand containing the sequence of a SEQ ID NO has that sequence from 5′ to 3′ unless otherwise specified.

The term “3′”, when used directionally, generally refers to a region or position in a polynucleotide or oligonucleotide 3′ (toward the 3′ end of the nucleotide) from another region or position in the same polynucleotide or oligonucleotide. The term “3′ end” generally refers to the 3′ terminal nucleotide of the component oligonucleotides.

The term “5′”, when used directionally, generally refers to a region or position in a polynucleotide or oligonucleotide 5′ (toward the 5′end of the nucleotide) from another region or position in the same polynucleotide or oligonucleotide. As used herein, the term “5′ end” generally refers to the 5′ terminal nucleotide of the component oligonucleotide.

The term “3′ domain” is generally at least 10 to nucleotides in length and refers to the first 10 to 12 nucleotides of the antisense oligonucleotide as measured from the 3′ end.

The term “5′ domain” is generally 2 to 15 nucleotides in length and refers to the 11^{th} through the 25^{th} nucleotides, 12^{th} through the 25^{th} nucleotides, or 13^{th} through the 25^{th} nucleotides of the antisense oligonucleotide as measured from the 3′ end depending on the length of the 3′ domain. For example, in some embodiments wherein the 3′ domain antisense oligonucleotide is 10 nucleotides in length, the term “5′ domain” generally refers to the 11^{th} through the 22^{th} nucleotides as measured from the 3′ end; the 11^{th} through the 21^{st} nucleotides as measured from the 3′ end; the 11^{th} through the 20^{th} nucleotides as measured from the 3′ end; the 11^{th} through the 19^{th} nucleotides as measured from the 3′ end; the 11^{th} through the 18^{th} nucleotides as measured from the 3′ end; the 11^{th} through the 17^{th} nucleotides as measured from the 3′ end; the 11^{th} through the 16^{th} nucleotides as measured from the 3′ end; the 11^{th} through the 15^{th} nucleotides as measured from the 3′ end; or the 11^{th} through the 14^{th} nucleotides as measured from the 3′ end.

The term “about” generally means that the exact number is not critical. Thus, oligonucleotides having one or two fewer nucleoside residues, or from one to several additional nucleoside residues are contemplated as equivalents of each of the embodiments described above.

“Antisense activity” means any detectable or measurable activity attributable to the hybridization of antisense oligonucleotide compound to its target nucleic acid. In certain embodiments, antisense activity is a decrease in the amount or expression of a target nucleic acid or protein encoded by such target nucleic acid. In certain embodiments, antisense activity is the modulation of splicing.

“Antisense inhibition” means reduction of target nucleic acid levels or target protein levels in the presence of an antisense oligonucleotide complementary to a target nucleic acid as compared to target nucleic acid levels or target protein levels in the absence of the antisense oligonucleotide.

“Antisense oligonucleotide” means a single-stranded oligonucleotide having a nucleobase sequence that permits hybridization to a corresponding region or segment of a target nucleic acid.

The term “co-administration” or “co-administered” generally refers to the administration of at least two different substances. Co-administration refers to simultaneous administration, as well as temporally spaced order of up to several days apart, of at least two different substances in any order, either in a single dose or separate doses.

The term “in combination with” generally means administering an oligonucleotide-based compound according to the invention and another agent useful for treating a disease or condition that does not abolish the activity of the compound in the course of treating a patient. Such administration may be done in any order, including simultaneous administration, as well as temporally spaced order from a few seconds up to several days apart. Such combination treatment may also include more than a single administration of the compound according to the invention and/or independently the other agent. The administration of the compound according to the invention and the other agent may be by the same or different routes.

The term “individual” or “subject” or “patient” generally refers to a mammal, such as a human. The term “mammal” is expressly intended to include warm blooded, vertebrate animals, including, without limitation, humans, non-human primates, rats, mice, cats, dogs, horses, cattle, cows, pigs, sheep and rabbits. As used herein, “individual in need thereof” refers to a human or non-human animal selected for treatment or therapy that is in need of such treatment or therapy.

As used herein, “inhibiting the expression or activity” refers to a reduction or blockade of the expression or activity of a RNA or protein and does not necessarily indicate a total elimination of expression or activity.

The term “nucleoside” generally refers to compounds consisting of a sugar, usually ribose, deoxyribose, pentose, arabinose or hexose, and a purine or pyrimidine base. For purposes of the invention, a base is considered to be non-natural if it is not guanine, cytosine, adenine, thymine or uracil and a sugar is considered to be non-natural if it is not β-ribo-furanoside or 2′-deoxyribo-furanoside.

The term “nucleotide” generally refers to a nucleoside comprising a phosphorous-containing group attached to the sugar. As used herein, “linked nucleosides” may or may not be linked by phosphate linkages and thus includes, but is not limited to, “linked nucleotides.” As used herein, “linked nucleosides” are nucleosides that are connected in a continuous sequence (i.e., no additional nucleosides are present between those that are linked).

The term “nucleic acid” encompasses a genomic region or an RNA molecule transcribed therefrom. In some embodiments, the nucleic acid is mRNA. In some embodiments, the nucleic acid is microRNA. In some embodiments, the nucleic acid is ncRNA.

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified. As used herein, “nucleobase sequence” means the order of contiguous nucleobases independent of any sugar, linkage, or nucleobase modification.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides comprise a modified sugar moiety and/or a modified nucleobase.

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA). In certain embodiments, an oligonucleotide comprises only unmodified ribonucleosides (RNA) and/or unmodified deoxyribonucleosides (DNA). In certain embodiments, an oligonucleotide comprises one or more modified nucleosides.

As used herein, “modified oligonucleotide” means an oligonucleotide comprising at least one modified nucleoside and/or at least one modified sugar.

As used herein “internucleoside linkage” means a covalent linkage between adjacent nucleosides in an oligonucleotide. As used herein “naturally occurring internucleoside linkage” means a 3′ to 5′ phosphodiester linkage. As used herein, “modified internucleoside linkage” means any internucleoside linkage other than a naturally occurring internucleoside linkage.

The phrase “an oligonucleotide that is complementary to a single-stranded RNA sequence” and the like, means that the oligonucleotide forms a sufficient number of hydrogen bonds through Watson-Crick interactions of its nucleobases with nucleobases of the single-stranded RNA sequence to form a double helix with the single-stranded RNA sequence under physiological conditions. This is in contrast to oligonucleotides that form a triple helix with a double-stranded DNA or RNA through Hoogsteen hydrogen bonding.

As used herein, “chemical modification” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

The term “complementary” is intended to mean an oligonucleotide that binds to the nucleic acid sequence under physiological conditions, for example, by Watson-Crick base pairing (interaction between oligonucleotide and single-stranded nucleic acid) or by Hoogsteen base pairing (interaction between oligonucleotide and double-stranded nucleic acid) or by any other means, including in the case of an oligonucleotide, binding to RNA and causing pseudoknot formation. Binding by Watson-Crick or Hoogsteen base pairing under physiological conditions is measured as a practical matter by observing interference with the function of the nucleic acid sequence.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Hybridization” means the annealing of complementary nucleic acid molecules. In certain embodiments, complementary nucleic acid molecules include an antisense compound and a target nucleic acid.

The term “pharmaceutically acceptable” means a non-toxic material that does not interfere with the effectiveness of a compound according to the invention or the biological activity of a compound according to the invention.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

The term “prophylactically effective amount” generally refers to an amount sufficient to prevent or reduce the development of an undesired biological effect.

As used herein, “RNase H based antisense compound” means an antisense compound wherein at least some of the antisense activity of the antisense compound is attributable to hybridization of the antisense compound to a target nucleic acid and subsequent cleavage of the target nucleic acid by RNase H.

As used herein, “sugar moiety” means a naturally occurring sugar moiety or a modified sugar moiety of a nucleoside. As used herein, “naturally occurring sugar moiety” means a ribofuranosyl as found in naturally occurring RNA or a deoxyribofuranosyl as found in naturally occurring DNA. As used herein, “modified sugar moiety” means a substituted sugar moiety or a sugar surrogate, such as, but not limited to 2′ modified sugars or constrained sugars.

The term “therapeutically effective amount” or “pharmaceutically effective amount” generally refers to an amount sufficient to affect a desired biological effect, such as a beneficial result, including, without limitation, prevention, diminution, amelioration or elimination of signs or symptoms of a disease or disorder. Thus, the total amount of each active component of the pharmaceutical composition or method is sufficient to show a meaningful patient benefit, for example, but not limited to, healing of chronic conditions characterized by immune stimulation. Thus, a “pharmaceutically effective amount” will depend upon the context in which it is being administered. A pharmaceutically effective amount may be administered in one or more prophylactic or therapeutic administrations. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “treatment” generally refers to an approach intended to obtain a beneficial or desired result, which may include alleviation of symptoms, or delaying or ameliorating a disease progression.

The term “gene expression” generally refers to process by which information from a gene is used in the synthesis of a functional gene product, which may be a protein. The process may involve transcription, RNA splicing, translation, and post-translational modification of a protein, and may include mRNA, pre-mRNA, noncoding RNA, snoRNA, ribosomal RNA, and other templates for protein synthesis.

“Targeting” or “targeted” means the process of design and selection of an antisense oligonucleotide that will specifically hybridize to a target nucleic acid and induces a desired effect. “Target gene”, “target allele”, “target nucleic acid,” “target RNA,” “target mRNA,” and “target RNA transcript” all refer to a nucleic acid whose expression is to be selectively inhibited or silenced. A “target allele” is an allele whose expression is to be selectively inhibited or silenced. “Target segment”, “target region”, and “target site” all refer to the sequence of nucleotides of a target nucleic acid to which antisense oligonucleotide is targeted.

A target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region.

### Oligonucleotide Compounds

The invention provides antisense oligonucleotides complementary to a nucleotide sequence of a target RNA, wherein the antisense oligonucleotides comprise a 3′ domain and a 5′ domain. The overall goal is to design an antisense which is potent and has specificity for its target RNA. In embodiments, the antisense oligonucleotide has two domains. The first region referred to as 3′-domain and is 10 to 12 nucleotides in length. The second region referred to as 5′-domain is 2 to 15 nucleotides in length and is contiguous with the 3′ domain.

The 3′-domain hybridizes to target RNA and activates RNase H. In embodiments, the 3′-domain is the first 10 to 12 nucleotides as measured from the 3′ end. In embodiments, the 3′-domain is the first 10 nucleotides as measured from the 3′ end. In embodiments, the 3′-domain is the first 11 nucleotides as measured from the 3′ end. In embodiments, the 3′-domain is the first 12 nucleotides as measured from the 3′ end. Each of the nucleotides of the 3′ domain comprise a deoxyribonucleotide and a phosphodiester or phosphothioate internucleotide linkage or combinations thereof. The nucleotides of the 3′-domain comprise natural deoxyribose sugar and phosphorothioate, phosphodiester or other phosphorus-based linkages or combinations thereof, which are known to activate RNase H.

The 5′-domain hybridizes to the target RNA but does not allow RNase H to excise the target RNA in this domain. The term “5′ domain” is generally 2 to 15 nucleotides in length and refers to the 11^{th} through the 25^{th} nucleotides, 12^{th} through the 25^{th} nucleotides, or 13^{th} through the 25^{th} nucleotides of the antisense oligonucleotide as measured from the 3′ end depending on the length of the 3′ domain. In some embodiments, the “5′ domain” is generally 5 to 15 nucleotides in length.

The 5′ domain comprises nucleotides having non-RNase H activating modifications such as modified sugars and/or modified backbones, which do not activate RNase H. In some embodiments, the 5′ domain comprises nucleotides comprising a modified sugar. In some embodiments, the 5′ domain comprises nucleotides comprising a modified backbone. In some embodiments, the 5′ domain comprises nucleotides comprising both a modified sugar and modified backbone. In embodiments, the modified backbone is a nonphosphorus-based backbone.

This design of antisense allows for targeted RNA cleavage at the specific sites towards the 5′ end of 3′-domain.

If there are mismatches in the targeted RNA in this region, efficiency of cleavage is reduced, allowing to obtain allele specificity. This design also allows to mitigate off-target effects as degradation of 3′-region from the 3′-end reduces the optimal length required for RNase H mediated cleavage thereby mitigating off target effects, contrary to what is observed with, for example, the gapmer design, which protects at both ends of an antisense oligonucleotide thereby increasing the in vivo persistence and availability of antisense to continue to bind and cleave RNA targets.

The antisense oligonucleotides of the invention are pharmaceutically acceptable. The antisense oligonucleotides of the invention are injectable. In certain embodiments, the antisense oligonucleotide is useful in methods for decreasing mRNA and/or protein expression. In certain embodiments, the antisense oligonucleotide is useful for treating, preventing, or ameliorating a disease associated with mRNA and/or protein expression. In some embodiments, the target RNA may be a pre, mRNA, mRNA noncoding RNA (ncRNA) and microRNA.

The invention provides an antisense oligonucleotide compound 17 to 25 nucleotides in length comprising at least 12 contiguous nucleobases complementary to an equal length portion of a target RNA sequence, wherein the antisense oligonucleotide compound comprises a 3′ domain and a 5′ domain, wherein the 3′ domain is 10 to 12 nucleotides in length and each nucleotide comprises a deoxyribonucleotide and a phosphodiester or phosphothioate internucleotide linkage or combinations thereof; and wherein the 5′ domain is 5 to 15 nucleotides in length, and wherein the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone.

In some embodiments, the 3′ domain comprises nucleotides at positions 1, 2, 3, 4, 5, 6, 7, 8, 9, 10, 11, and 12 from the 3′ end. In some embodiments, the 3′ domain comprises nucleotides at positions 1, 2, 3, 4, 5, 6, 7, 8, 9, 10, and 11 from the 3′ end. In some embodiments, the 3′ domain comprises nucleotides at positions 1, 2, 3, 4, 5, 6, 7, 8, 9, and 10 from the 3′ end.

In some embodiments, when the 3′ domain of the antisense oligonucleotide is 12 nucleotides in length, the antisense oligonucleotides of the invention are represented by Formula (I):

5′-NₘN₁₄N₁₃N₁₂N₁₁N₁₀N₉N₈N₇N₆N₅N₄N₃N₂N₁-3′

wherein
N is any nucleotide;
N₁₃ through Nₘ comprises the 5′ domain;
N₁ through N₁₂ comprises the 3′ domain; and
m is selected from 0, 1, 2, 3, 4, 5, 6, 7, 8, 9, 10, or 11.

In some embodiments, when the 3′ domain of the antisense oligonucleotide is 11 nucleotides in length, the antisense oligonucleotides of the invention are represented by Formula (Ia):

5′-NₘN₁₄N₁₃N₁₂N₁₁N₁₀N₉N₈N₇N₆N₅N₄N₃N₂N₁-3′

wherein
N is any nucleotide;
N₁₂ through Nₘ comprises the 5′ domain;
N₁ through N₁₁ comprises the 3′ domain; and
m is selected from 0, 1, 2, 3, 4, 5, 6, 7, 8, 9, 10, or 11.

In some embodiments, the antisense oligonucleotides of the invention are represented by Formula (Ib):

5′-NₘN₁₄N₁₃N₁₂N₁₁N₁₀N₉N₈N₇N₆N₅N₄N₃N₂N₁-3′

wherein
N is any nucleotide;
N₁₁ through Nₘ comprises the 5′ domain;
N₁ through N₁₀ comprises the 3′ domain; and
m is selected from 0, 1, 2, 3, 4, 5, 6, 7, 8, 9, 10, or 11.

In some embodiments, m is 0. In some embodiments, m is selected from 1, 2, 3, 4, 5, 6, or 7. In some embodiments, m is selected from 1, 2, 3, 4, 5, or 6. In some embodiments, m is selected from 1, 2, 3, 4, or 5. In some embodiments, m is selected from 1, 2, 3, or 4. In some embodiments, m is selected from 1, 2, or 3. In some embodiments, m is 1. In some embodiments, m is 2. In some embodiments, m is 3. In some embodiments, m is 4. In some embodiments, m is 5. In some embodiments, m is 6. In some embodiments, m is 7.

In some embodiments, m is 8. In some embodiments, m is 9. In some embodiments, m is 10. In some embodiments, m is 11.

Without wishing to be bound to any particular theory, the 9^{th} position from the 3′ end of the antisense oligonucleotide is important. RNase H makes a major cut of the antisense oligonucleotide-target RNA complex at around the 9^{th} position. RNase H can make a second important cut at around the 17^{th} position. Modification or mismatch of the nucleotide of the antisense oligonucleotide at the 9^{th} position can interfere with RNase H activity. Modification or mismatch of the 8^{th} and/or 10^{th} positions from the 3′ end of the antisense oligonucleotide can also interfere with RNase H but to a lesser extent than the 9^{th} position. Additionally, an antisense oligonucleotide 17 nucleotides in length may avoid a non-specific second cut by RNase H.

Certain embodiments provide an antisense oligonucleotide comprising a 3′ domain and a 5′ domain, wherein the each nucleotide of the 5′ domain independently comprises a natural sugar, a natural internucleotide linkage, a natural nucleobase, a modified internucleotide linkage, a modified sugar, a modified nucleobase or combinations thereof, provided that at least one of the nucleotides of the 5′ domain comprises a modified internucleotide linkage (i.e., backbone) and/or a modified sugar; and wherein the nucleotides of the 3′ domain comprise a natural sugar and a natural nucleobase and either a natural internucleotide linkage or a modified phosphorus-based internucleotide linkage. Certain embodiments provide an antisense oligonucleotide comprising a 5′ domain, a 3′ domain, and a 5′ end blocking agent, wherein the each nucleotide of the 5′ domain independently comprises a natural sugar, a natural internucleotide linkage, a natural nucleobase, a modified internucleotide linkage, a modified sugar, a modified nucleobase or combinations thereof, provided that at least one of the nucleotides of the 5′ domain comprises a modified internucleotide linkage and/or a modified sugar; and wherein the nucleotides of the 3′ domain comprise a natural sugar and a natural nucleobase and either a natural internucleotide linkage or a modified phosphorus-based internucleotide linkage.

As used here, the term “3′ domain” refers to the nucleotides at positions 1, 2, 3, 4, 5, 6, 7, 8, 9, 10, 11, and 12 of the antisense oligonucleotide compound (position 1 is the 3′ end). In embodiments, the term “3′ domain” refers to the nucleotides at positions 1, 2, 3, 4, 5, 6, 7, 8, 9, 10, and 11 of the antisense oligonucleotide compound (position 1 is the 3′ end). herein embodiments, the term “3′ domain” refers to the nucleotides at positions 1, 2, 3, 4, 5, 6, 7, 8, 9, and 10 of the antisense oligonucleotide compound (position 1 is the 3′ end). In some embodiments, the nucleobases and sugars of the nucleotides of the 3′ domain of the antisense oligonucleotide according to the invention are unmodified. In this respect, the nucleobases and sugars of the nucleotides of the 3′ domain of the antisense oligonucleotide according to the invention are naturally occurring.

In some embodiments, the 3′ domain of the antisense oligonucleotide according to the invention has a modified nucleobase that does not interfere with RNase H activity. The modified nucleobase can be any suitable modified nucleobase as described herein. In some embodiments, the 3′ domain of the antisense oligonucleotide according to the invention has a modified nucleobase at one or more positions. In some embodiments, nucleotides at positions 8, 9, and 10 from the 3′ end are unmodified.

As used here, the term “5′ domain” refers to the nucleotides beginning at the first nucleotide following the 3′ domain and goes to the 5′ end. In some embodiments, the term “5′ domain” refers to the nucleotides beginning at position 11 of the antisense oligonucleotide compound (position 1 is the 3′ end) and goes to the 5′ end. The nucleotides of the 5′ domain comprise unmodified nucleotides, modified nucleotides, or combinations thereof provided that the 5′ domain comprises at least 1 modified nucleotide comprising a modified sugar and/or a modified backbone. In embodiments, the modified backbone is a nonphosphorus-based backbone.

For example, an antisense oligonucleotide compound that is 17 nucleotides in length may comprise a 3′ domain from position 1 to position 10 and a 5′ domain from position 11 to position 17. The designation of the modified nucleotide is position-specific, as opposed to nucleotide-specific.

In any of these embodiments it is contemplated that the 5′ domain comprises at least one nucleotide having a backbone modification or substitution and/or a sugar modification or substitution. In some embodiments, a nucleotide at one position within the 5′ domain, at some of the positions within the 5′ domain, or at all positions within the 5′ domain comprises a backbone modification or substitution and/or a sugar modification or substitution. In one embodiment, the 5′ domain comprises one nucleotide comprising a modified backbone and/or sugar. In one embodiment, the 5′ domain comprises at least two nucleotides comprising a modified backbone and/or sugar. In one embodiment, the 5′ domain comprises at least three nucleotides comprising a modified backbone and/or sugar. In one embodiment, the 5′ domain comprises at least four nucleotides comprising a modified backbone and/or sugar. In one embodiment, the 5′ domain comprises at least five nucleotides comprising a modified backbone and/or sugar. In one embodiment, the 5′ domain comprises at least six nucleotides comprising a modified backbone and/or sugar. In one embodiment, the 5′ domain comprises at least seven nucleotides comprising a modified backbone and/or sugar. In one embodiment, the 5′ domain comprises at least eight nucleotides comprising a modified backbone and/or sugar. In one embodiment, all of the nucleotides of the 5′ domain are nucleotides comprising a modified backbone and/or sugar. In some embodiments, the nucleotide at position 11 from the 3′ end is unmodified.

In embodiments where the 5′ domain comprises more than one modified nucleotide but where less than all nucleotides are modified, the modified nucleotides need not be consecutive. In some embodiments, the 5′ domain comprises modified nucleotides at positions 17 and 16 from the 3′ end. In some embodiments, the 5′ domain comprises modified nucleotides at positions 17, 16, and 15 from the 3′ end. In some embodiments, the 5′ domain comprises modified nucleotides at positions 17, 16, and 14 from the 3′ end. In some embodiments, the 5′ domain comprises modified nucleotides at positions 17, 16, 14, and 13 from the 3′ end. In some embodiments, the 5′ domain comprises modified nucleotides at positions 17, 16, 14, and 12 from the 3′ end. In some embodiments, the 5′ domain comprises modified nucleotides at positions 17, 16, 14, 12, and 11 from the 3′ end. For example, an antisense oligonucleotide that is 17 nucleotides in length and comprises 3 modified nucleotides within the 5′ domain, may comprise the modified nucleotide at positions 17, 16, and 15, or at positions 17, 16, and 14, or at positions 17, 16, and 12, or at any other possible combination derivable therein. In a further example, an antisense oligonucleotide that is 17 nucleotides in length and comprises 5 modified nucleotides within the 5′ domain, may comprise the modified nucleotide at positions 17, 16, 15, 14, and 13, or at positions 17, 16, 14, 13, and 12, or at positions 17, 15, 14, 13, and 12, or at positions 17, 16, 14, 12, and 11, or at any other possible combination derivable therein.

It is specifically contemplated that embodiments discussed herein, in the context of a specific nucleotide and position, may be implemented with respect to a position relative to the 3′ end. For example, an antisense oligonucleotide with a modified nucleotide at position 13 refers to an antisense oligonucleotide having a modified nucleotide at position 13 from the 3′ end of the antisense oligonucleotide.

Certain embodiments provide an antisense oligonucleotide wherein the antisense oligonucleotide is single-stranded. Certain embodiments provide an antisense oligonucleotide wherein the antisense oligonucleotide comprises unmodified nucleotides.

In some embodiments, the invention provides an antisense oligonucleotide compound 14 to 25 nucleotides in length nucleotides in length comprising at least 12 contiguous nucleobases complementary to an equal length portion of a target sequence, wherein the antisense oligonucleotide compound comprises a 3′ domain and a 5′ domain as described herein. In some embodiments, the 3′ domain comprises nucleotides at positions 1, 2, 3, 4, 5, 6, 7, 8, 9, 10, 11, and 12 from the 3′ end. In some embodiments, the 3′ domain comprises nucleotides at positions 1, 2, 3, 4, 5, 6, 7, 8, 9, 10, and 11 from the 3′ end. In some embodiments, the 3′ domain comprises nucleotides at positions 1, 2, 3, 4, 5, 6, 7, 8, 9, and 10 from the 3′ end.

In some embodiments, the 3′ domain comprises nucleotides at positions 1, 2, 3, 4, 5, 6, 7, 8, 9, and 10 from the 3′ end. In some embodiments, the nucleotides of the 3′ domain comprise a natural nucleobase and a natural sugar. In some embodiments, at least one of the nucleotides of the 3′ domain comprises a modified base. In some embodiments, nucleotides at positions 8, 9, and 10 from the 3′ end are unmodified. In some embodiments, the 5′ domain comprises the nucleotides at positions 11 to 25 from the 3′ end. In some embodiments, the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone. In some embodiments, the modified backbone is a nonphosphorus-based backbone. In some embodiments, wherein the antisense oligonucleotide comprises 18 or more nucleotides in length, the antisense oligonucleotide further comprises a 5′ end blocking agent as discussed herein.

In some embodiments, the invention provides an antisense oligonucleotide compound 17 nucleotides in length nucleotides in length comprising at least 12 contiguous nucleobases complementary to an equal length portion of a target sequence, wherein the antisense oligonucleotide compound comprises a 3′ domain and a 5′ domain as described herein. In some embodiments, the 3′ domain comprises nucleotides at positions 1, 2, 3, 4, 5, 6, 7, 8, 9, and 10 from the 3′ end. In some embodiments, the nucleotides of the 3′ domain comprise a natural nucleobase and a natural sugar. In some embodiments, at least one of the nucleotides of the 3′ domain comprises a modified base. In some embodiments, nucleotides at positions 8, 9, and 10 from the 3′ end are unmodified. In some embodiments, the 5′ domain comprises the nucleotides at positions 11, 12, 13, 14, 15, 16 and 17 from the 3′ end. In some embodiments, the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone. In some embodiments, the modified backbone is a nonphosphorus-based backbone.

In some embodiments, the invention provides an antisense oligonucleotide compound 18 to 25 nucleotides in length nucleotides in length comprising at least 12 contiguous nucleobases complementary to an equal length portion of a target sequence, wherein the antisense oligonucleotide compound comprises a 3′ domain, a 5′ domain and a 5′ end blocking agent as described herein. In some embodiments, the 3′ domain comprises nucleotides at positions 1, 2, 3, 4, 5, 6, 7, 8, 9, and 10 from the 3′ end. In some embodiments, the nucleotides of the 3′ domain comprise a natural nucleobase and a natural sugar. In some embodiments, at least one of the nucleotides of the 3′ domain comprises a modified base. In some embodiments, nucleotides at positions 8, 9, and 10 from the 3′ end are unmodified. In some embodiments, the 5′ domain comprises the nucleotides at positions 18 to 25 from the 3′ end. In some embodiments, the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone. In some embodiments, the modified backbone is a nonphosphorus-based backbone.

In some embodiments, the antisense oligonucleotide compound is 18 nucleotides in length. In some embodiments, the antisense oligonucleotide compound is 19 nucleotides in length. In some embodiments, the antisense oligonucleotide compound is 20 nucleotides in length. In some embodiments, the antisense oligonucleotide compound is 21 nucleotides in length. In some embodiments, the antisense oligonucleotide compound is 22 nucleotides in length. In some embodiments, the antisense oligonucleotide compound is 23 nucleotides in length. In some embodiments, the antisense oligonucleotide compound is 24 nucleotides in length. In some embodiments, the antisense oligonucleotide compound is 25 nucleotides in length.

In some embodiments, the antisense oligonucleotides of the invention may be at least 14 nucleotides in length, for example between 14 to 25 nucleotides in length. Thus, the antisense oligonucleotides of the invention may be 14, 15, 16, 17, 18, 19, 20, 21, 22, 23, 24, or 25 nucleotides in length. The antisense oligonucleotides of the invention may be 17, 18, 19, 20, 21, or 22 nucleotides in length. In some embodiments, the antisense oligonucleotides of the invention may be 17 nucleotides in length. The antisense oligonucleotides of the invention may be 18 nucleotides in length. The antisense oligonucleotides of the invention may be 19 nucleotides in length. The antisense oligonucleotides of the invention may be 20 nucleotides in length. The antisense oligonucleotides of the invention may be 21 nucleotides in length. The antisense oligonucleotides of the invention may be 22 nucleotides in length.

The antisense oligonucleotides of the invention may be 23 nucleotides in length. The antisense oligonucleotides of the invention may be 24 nucleotides in length. The antisense oligonucleotides of the invention may be 25 nucleotides in length.

As used herein, the natural or unmodified bases in RNA are adenine (A) and guanine (G), and the pyrimidine bases cytosine (C) and uracil (U) (DNA has thymine (T)). In contrast, modified bases, also referred to as heterocyclic base moieties, include other nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

In certain embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine. Further modified nucleobases include tricyclic pyrimidines such as phenoxazine cytidine ([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. In certain embodiments, the modified nucleobase is a 5-methylcytosine.

Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an antisense oligonucleotide may have one or more nucleotides with a sugar modification at one position and also one or more nucleotides with a sugar modification at a different position.

Sugar modifications contemplated in an antisense oligonucleotide include, but are not limited to, a sugar substituent group selected from: OH; F; O-, S-, or N-alkyl; O-, S-, or N-alkenyl; O-, S or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In some embodiments, these groups may be chosen from: O(CH₂)ₓOCH₃, O((CH₂)ₓO)_{y}CH₃, O(CH₂)ₓNH₂, O(CH₂)ₓCH₃, O(CH₂)ₓONH₂, and O(CH₂)ₓON((CH₂)ₓCH₃)₂, where x and y are independently from 1 to 10.

In some embodiments, the modified sugar comprises a substituent group selected from the following: C₁ to C₁₀ lower alkyl, substituted lower alkyl, alkenyl, alkynyl, alkaryl, aralkyl, O-alkaryl or O-aralkyl, SH, SCH₃, Cl, Br, CN, OCN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an antisense oligonucleotide, or a group for improving the pharmacodynamic properties of an antisense oligonucleotide, and other substituents having similar properties. In one embodiment, the modification includes 2′-methoxyethoxy (2′-O—CH₂CH₂OCH₃, which is also known as 2′-O-(2-methoxyethyl) or 2′-MOE) (Martin et al., 1995), that is, an alkoxyalkoxy group. Another modification includes 2′-dimethylaminooxyethoxy, that is, a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃)₂.

Additional sugar substituent groups include allyl (—CH₂—CH═CH₂), —O-allyl CH₂—CH═CH₂), methoxy (—O—CH₃), aminopropoxy (—OCH₂CH₂CH₂NH₂), and fluoro (F). Sugar substituent groups on the 2′ position (2′-) may be in the arabino (up) position or ribo (down) position. One 2′-arabino modification is 2′-F. Other similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics, for example, cyclobutyl moieties, in place of the pentofuranosyl sugar.

Examples of U.S. patents that disclose the preparation of modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, which are herein incorporated by reference in its entirety.

Representative sugar substituent groups include groups described in U.S. Patent Application Publication 2005/0261218, which is hereby incorporated by reference. In particular embodiments, the sugar modification is a 2′-O-Me modification, a 2′ F modification, a 2′ H modification, a 2′ amino modification, a 4′ thioribose modification or a phosphorothioate modification on the carboxy group linked to the carbon at position 6′, or combinations thereof.

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al, U.S. Pat. No. 7,569,686, Swayze et al., U.S. Pat. No. 7,741,457, and Swayze et al, U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al, U.S. Pat. No. 8,278,425), 4′-CH₂—O—N(CH₃)-2′ (see, e.g., Allerson et al., U.S. Pat. No. 7,696,345 and Allerson et al, U.S. Pat. No. 8,124,745), 4′-CH₂—C(H)(CH₃)-2′ (see, e.g., Zhou, et al, J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ and R_{b} is, independently, H, a protecting group, or C₁-C₁₂ alkyl (see, e.g., Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —S(═O)ₓ—, and —N(Rₐ)—;

wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N3, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J Org. Chem., 1998, 63, 10035-10039; Srivastava et al., J Am. Chem. Soc, 20017, 129, 8362-8379; Wengel et al., U.S. Pat. No. 7,053,207; Imanishi et al., U.S. Pat. No. 6,268,490; Imanishi et al., U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

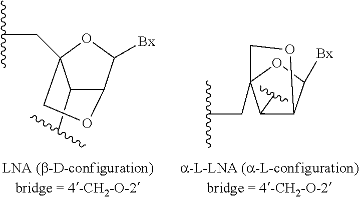

α-L-methyleneoxy (4′-CH₂-0-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position. In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

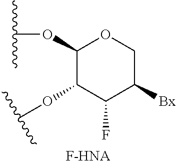

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

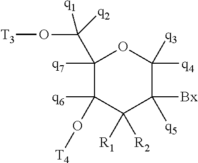

wherein, independently, for each of said modified THP nucleoside:

Bx is a nucleobase moiety;

T3 and T4 are each, independently, an internucleoside linking group linking the modified THP nucleoside to the remainder of an oligonucleotide or one of T3 and T4 is an internucleoside linking group linking the modified THP nucleoside to the remainder of an oligonucleotide and the other of T3 and T4 is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

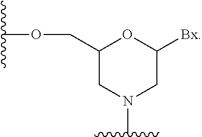

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

The nucleoside residues of the antisense oligonucleotides can be coupled to each other by any of the numerous known internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative nonphosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Methods of preparation of phosphorous-containing and non-phosphorous-containing internucleoside linkages are well known to those skilled in the art.

Such internucleoside linkages include, without limitation, phosphodiester, phosphorothioate, phosphorodithioate, methylphosphonate, alkylphosphonate, alkylphosphonothioate, phosphotriester, phosphoramidate, siloxane, carbonate, carboalkoxy, acetamidate, carbamate, morpholino, borano, thioether, bridged phosphoramidate, bridged methylene phosphonate, bridged phosphorothioate, and sulfone internucleoside linkages. In some embodiments, the synthetic antisense oligonucleotides of the invention may comprise combinations of internucleotide linkages. In some embodiments, the synthetic antisense oligonucleotides of the invention may comprise combinations of phosphorothioate and phosphodiester internucleotide linkages. In some embodiments more than half but less that all of the internucleotide linkages are phosphorothioate internucleotide linkages. In some embodiments all of the internucleotide linkages are phosphorothioate internucleotide linkages.

Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration.

In certain embodiments, the phosphorothioate linkages may be mixed Rp and Sp enantiomers, or they may be made stereoregular or substantially stereoregular in either Rp or Sp form. In embodiments where the linkages are mixed Rp and Sp enantiomers, the Rp and Sp forms may be at defined places within the antisense oligonucleotide or randomly placed throughout the oligonucleotide.

In some embodiments, the backbone of the 3′ domain comprises phosphorothioate internucleotide linkages and the backbone of the 5′ domain comprises phosphodiester internucleotide linkages, phosphorothioate internucleotide linkages, or combinations thereof. In some embodiments, the backbone of the 3′ domain and 5′ domain comprises phosphorothioate internucleotide linkages.

In some embodiments, where the antisense oligonucleotide comprises 18 or more nucleotides in length, the antisense oligonucleotide further comprises a 5′ end blocking agent. As used herein, a 5′ end blocking agent refers a modification or motif linked to the 5′ end of the antisense oligonucleotide. Without wishing to be bound to a particular theory, the presence of a 5′ end blocking agent may prevent excision of the bound antisense oligonucleotide by RNase H beyond the 11^{th} nucleotide from the 3′ end and, for example, target the RNA molecule between the 17^{th}, and 18^{th} positions from the 3′ end. Such 5′ end blocking agents may promote specificity, increased potency, in vivo stability and less off-target activity. 5′ end blocking agents include, but are not limited to, a non-ionic backbone modification, non-complementary overhanging nucleotides, 2′-substituted ribonucleotides, locked nucleic acid (LNA) nucleotides, acyclic nucleotides, inverted deoxyabasic moieties, a conjugate, a non-nucleotide moiety.

In some embodiments, the 5′ end blocking agent comprises one or more non-natural nucleotides. In some embodiments, the 5′ end blocking agent comprises a non-natural nucleotide. In some embodiments, such non-natural nucleotides are added to the 5′ end of the antisense oligonucleotide. For example, in some embodiments, the 5′ end blocking agent may comprise from about 1 to about 6 non-natural nucleotides added to the 5′ end of the antisense oligonucleotide. In some embodiments, the 5′ end blocking agent may be about 1 to about 3 non-natural nucleotides added to the 5′ end of the antisense oligonucleotide. In some embodiments, the 5′ end blocking agent may be 1 or 2 non-natural nucleotides added to the 5′ end of the antisense oligonucleotide. For the sake of clarity, an antisense oligonucleotide comprising a 5′ end blocking agent according to the invention may be, for example, 17 nucleotides in length and comprise a 5′ end blocking agent comprising 3 non-natural nucleotides so that the total length of the antisense oligonucleotide is 20 nucleotides. In this example, position 4 (as determined from the 5′ end) is the 5′ end of the antisense oligonucleotide and positions 1 to 3 make up the “5′ end blocking agent”. In some embodiments, the non-natural nucleotides of the 5′ end blocking agent may be complementary to the target RNA. In some embodiments, the non-natural nucleotides of the 5′ end blocking agent are not complementary to the target RNA.

In some embodiments, the 5′ end blocking agent comprises one or more nucleotides having a non-ionic backbone. In some embodiments, the 5′ end blocking agent comprises nucleotides having a non-ionic backbone. In some embodiments, such nucleotides having a non-ionic backbone are added to the 5′end of the antisense oligonucleotide. For example, in some embodiments, the 5′ end blocking agent may comprise from about 1 to about 6 nucleotides having a non-ionic backbone added to the 5′ end of the antisense oligonucleotide. In some embodiments, the 5′ end blocking agent may be about 1 to about 3 nucleotides having a non-ionic backbone added to the 5′ end of the antisense oligonucleotide. In some embodiments, the 5′ end blocking agent may be 1 or 2 nucleotides having a non-ionic backbone added to the 5′ end of the antisense oligonucleotide. Such a backbone modification may include, but is not limited to, methylphosphonate or phosphoramidite. In some embodiments, the nucleotides having a non-ionic backbone of the 5′ end blocking agent may be complementary to the target RNA. In some embodiments, the nucleotides having a non-ionic backbone of the 5′ end blocking agent are not complementary to the target RNA.

In some embodiments, the 5′ end blocking agent comprises one or more overhanging nucleotides, which are not complementary to the nucleotide sequence of the target RNA. In some embodiments, the 5′ end blocking agent comprises from about one to about six overhanging nucleotides, which are not complementary to the nucleotide sequence of the target RNA. In some embodiments, the 5′ end blocking agent may comprise one overhanging nucleotide. In some embodiments, the 5′ end blocking agent may comprise two overhanging nucleotides. In some embodiments, the 5′ end blocking agent may comprise three overhanging nucleotides. In some embodiments, the 5′ end blocking agent may comprise four overhanging nucleotides.

In some embodiments, the 5′ end blocking agent comprises a hairpin loop. A hairpin loop is an oligonucleotide that comprises nucleotides capable of intramolecular hybridization. In some embodiments, the hairpin loop of the 5′ end blocking agent comprises about 6 to about 25 (e.g., about 3, 4, 5, 6, 7, 8, 9, 10, 11, 12, 13, 14, 15, 16, 17, 18, 19, 20, 21, 22, 23, 24, or 25) nucleotides added to the 5′ end, wherein the additional nucleotide are not complementary to the target RNA and wherein at least a portion of the additional nucleotides have internal nucleobase complementarity and may fold in a manner as to produce a fully or partially double stranded structure.

In some embodiments, the 5′ end blocking agent comprises one or more 2′-substituted ribonucleotides. In some embodiments, the 5′ end blocking agent comprises about one to about six 2′-substituted ribonucleotides. For purposes of the invention, the term “2′-substituted” means substitution of the 2′-OH of the ribose molecule with, e.g., 2′-allyl, 2′-alkyl, 2′-aryl, 2′-O-allyl, 2′-O-alkyl, 2′-O-aryl, 2′-halo, or 2′-amino, but not with 2′-H, wherein allyl, aryl, or alkyl groups may be unsubstituted or substituted, e.g., with halo, hydroxy, trifluoromethyl, cyano, nitro, acyl, acyloxy, alkoxy, carboxyl, carbalkoxyl or amino groups. In some embodiments, the 5′ end blocking agent comprises one 2′ substituted ribonucleotide. In some embodiments, the 5′ end blocking agent comprises two 2′ substituted ribonucleotides. In some embodiments, the 5′ end blocking agent comprises three 2′ substituted ribonucleotides. In some embodiments, the 5′ end blocking agent comprises four 2′ substituted ribonucleotides. In some embodiments, the one or more 2′-substituted ribonucleotides of the 5′ end blocking agent may be fully or partially complementary to the target RNA. In some embodiments, the one or more 2′-substituted ribonucleotides of the 5′ end blocking agent are not complementary to the target RNA.

In some embodiments, the 5′ end blocking agent comprises one or more locked nucleic acid (LNA) nucleotides. In some embodiments, the 5′ end blocking agent comprises about 1, 2, 3, 4, 5, 6, 7, 8, 9, or 10 LNAs. In some embodiments, the one or more LNAs of the 5′ end blocking agent may be fully or partially complementary to the target RNA. In some embodiments, the one or more LNAs of the 5′ end blocking agent are not complementary to the target RNA.

In some embodiments, the 5′ end blocking agent comprises one or more acyclic nucleotides. In some embodiments, the 5′ end blocking agent comprises about 1, 2, 3, 4, 5, 6, 7, 8, 9, or 10 acyclic nucleotides. In some embodiments, the one or more acyclic nucleotides of the 5′ end blocking agent may be fully or partially complementary to the target RNA. In some embodiments, the one or more acyclic nucleotides of the 5′ end blocking agent are not complementary to the target RNA.

In some embodiments, the 5′ end blocking agent comprises one or more inverted deoxyabasic moieties. In some embodiments, the 5′ end blocking agent comprises about 1, 2, 3, 4, 5, 6, 7, 8, 9, or 10 inverted deoxyabasic moieties. In some embodiments, the one or more inverted deoxyabasic moieties of the 5′ end blocking agent may be fully or partially complementary to the target RNA. In some embodiments, the one or more inverted deoxyabasic moieties of the 5′ end blocking agent are not complementary to the target RNA.

In some embodiments, the 5′ end blocking agent comprises a conjugate moiety covalently attached to the 5′ end. Non-limiting examples of conjugates contemplated by the invention include conjugates and ligands described in Vargeese et al., U.S. Ser. No. 10/427,160, filed Apr. 30, 2003, incorporated by reference herein in its entirety.

In some embodiments, the 5′ end blocking agent comprises a non-nucleotide moiety. Non-limiting examples of non-nucleotide moieties include, but are not limited to, (glycerol, antibodies, lipids, fatty acids, peptides (such as those disclosed in https://www.ncbi.nlm.nih.gov/pubmed/30307373, which is incorporated herein by reference), or sugars (such as those disclosed in https://www.ncbi.nlm.nih.gov/pubmed/28988716, which is incorporated herein by reference).

In some embodiments, the 5′ end blocking agent is not a synthetic capping reagent used in the process of oligonucleotide synthesis. In some embodiments, the 5′ end blocking agent is not a crosslinking agent linking an oligonucleotide to a solid support.

The synthetic antisense compounds of the invention can be prepared by the art recognized methods such as phosphoramidite or H-phosphonate chemistry which can be carried out manually or by an automated synthesizer. The synthetic antisense compounds of the invention may also be modified in a number of ways without compromising their ability to hybridize to mRNA.

In some embodiments, the oligonucleotide-based compounds of the invention are synthesized by a linear synthesis approach.

At the end of the synthesis by either linear synthesis or parallel synthesis protocols, the oligonucleotide-based compounds of the invention may conveniently be deprotected with concentrated ammonia solution or as recommended by the phosphoramidite supplier, if a modified nucleoside is incorporated. The product oligonucleotide-based compounds is preferably purified by reversed phase HPLC, detritylated, desalted and dialyzed.

A non-limiting list of the antisense oligonucleotides of the invention are shown in Table 1. Unless otherwise noted, the antisense oligonucleotides have phosphorothioate (PS) backbone linkages. Those skilled in the art will recognize, however, that other linkages, based on phosphodiester or non-phosphodiester moieties may be included.

TABLE 1

| | Sequence | SEQ ID NO: | Target |
|---|---|---|---|
| 1 | 5′-TCTCCTCCATCAGCACC-3′ | 1 | PCSK9 |
| 2 | 5′-GGTCTCCTCCATCAGCACC-3′ | 2 | PCSK9 |
| 3 | 5′-UCUCCTCCATCAGCACC-3′ | 3 | PCSK9 |
| 4 | 5′-UCTCCUCCATCAGCACC-3′ | 4 | PCSK9 |
| 5 | 5′-UCUCCTCCCTCAGCACC-3′ | 5 | PCSK9 |
| 6 | 5′-UCTCCUCCCTCAGCACC-3′ | 6 | PCSK9 |
| 7 | 5′-TCTCCTCCCTCAGCACC-3′ | 7 | PCSK9 |
| 8 | 5′-TCTCCTCAATCAGCACC-3′ | 8 | PCSK9 |
| 9 | 5′-UCUCCTCCAUCAGCACC-3′ | 9 | PCSK9 |
| 10 | 5′-UCUCCTCCATCAGCACC-3′ | 10 | PCSK9 |
| 11 | 5′-UCTCCUCAATCAGCACC-3′ | 11 | PCSK9 |
| 12 | 5′-UCTCCUCCATCAGCACC-3′ | 12 | PCSK9 |
| 13 | 5′-UCTCCTCCATCAGCACC-3′ | 13 | PCSK9 |
| 14 | 5′-UcTcCuCCATCAGCACC-3′ | 14 | PCSK9 |
| 15 | 5′-UcTcCuCCCTCAGCACC-3′ | 15 | PCSK9 |

underlined = 2′-O-methyl phosphorothioate; underlined and lowercase 2′-O-methyl phosphodiester; italic = mismatch.

In certain embodiments, the target nucleic acid is the murine sequence of the target. In certain embodiments, the target nucleic acid is the human sequence of the target. In certain embodiments, the PCSK9 nucleic acid is the murine sequence set forth in GENBANK Accession No. NM_153565.2 (incorporated herein as SEQ ID NO: 16). In certain embodiments, the PCSK9 nucleic acid is the human sequence set forth in GENBANK Accession No. NM_174936.3 (incorporated herein as SEQ ID NO: 17).

The invention provides pharmaceutical compositions comprising the antisense oligonucleotides described herein and a pharmaceutically acceptable carrier. The term “carrier” generally encompasses any excipient, diluent, filler, salt, buffer, stabilizer, solubilizer, oil, lipid, lipid containing vesicle, microspheres, liposomal encapsulation, or other material for use in pharmaceutical formulations. It will be understood that the characteristics of the carrier, excipient or diluent will depend on the route of administration for a particular application. The preparation of pharmaceutically acceptable formulations containing these materials is described in, for example, Remington's Pharmaceutical Sciences, 18^{th} Edition, ed. A. Gennaro, Mack Publishing Co., Easton, Pa., 1990.

The composition may further comprise one or more other agents. Such agents may include but are not limited to, vaccines, antigens, antibodies, cytotoxic agents, chemotherapeutic agents (both traditional chemotherapy and modern targeted therapies), kinase inhibitors, allergens, antibiotics, agonist, antagonist, antisense oligonucleotides, ribozymes, RNAi molecules, siRNA molecules, miRNA molecules, aptamers, proteins, gene therapy vectors, DNA vaccines, adjuvants, co-stimulatory molecules or combinations thereof.

The nucleic acid sequence to which an oligonucleotide according to the invention is complementary will vary, depending upon the agent to be inhibited. For example, the antisense oligonucleotides according to the invention can have an oligonucleotide sequence complementary to a cellular gene or gene transcript, the abnormal expression or product of which results in a disease state. The nucleic acid sequences of several such cellular genes have been described in the art. Antisense oligonucleotides according to the invention can have any oligonucleotide sequence so long as the sequence is partially or fully complementary to a target RNA nucleotide sequence.

It is generally believed that the activity of an antisense oligonucleotide depends on the binding of the oligonucleotide to the target nucleic acid, thus disrupting the function of the target, either by hybridization arrest or by destruction of target RNA by RNase H. These mechanisms of action suggest that two parameters should be important to antisense oligonucleotide activity: duplex stability and RNase H activation. Duplex stability is important, since the oligonucleotide presumably must form a duplex with the target nucleic acid to act either by hybridization arrest or by RNase H-mediated target destruction. RNase H activation (the ability to activate RNase H when hybridized with target RNA) is implicated when the target nucleic acid is RNA, since such activation can lead to the effective destruction of the target RNA molecule.

In certain embodiments, oligomeric compounds of the present invention are antisense compounds. In such embodiments, the oligomeric compound is complementary to a target nucleic acid. In certain embodiments, a target nucleic acid is an RNA. In certain embodiments, a target nucleic acid is a non-coding RNA. In certain embodiments, a target nucleic acid encodes a protein. In certain embodiments, a target nucleic acid is selected from a mRNA, a pre-mRNA, a microRNA, a non-coding RNA, including small non-coding RNA, and a promoter-directed RNA.

In certain embodiments, the invention provides antisense oligonucleotides having a sequence complementary to a target nucleic acid. Such antisense compounds are capable of hybridizing to a target nucleic acid, resulting in at least one antisense activity. In certain embodiments, antisense compounds specifically hybridize to one or more target nucleic acid. In certain embodiments, a specifically hybridizing antisense compound has a nucleobase sequence comprising a region having sufficient complementarity to a target nucleic acid to allow hybridization and result in antisense activity and insufficient complementarity to any non-target so as to avoid or reduce non-specific hybridization to non-target nucleic acid sequences under conditions in which specific hybridization is desired (e.g., under physiological conditions for in vivo or therapeutic uses, and under conditions in which assays are performed in the case of in vitro assays). In certain embodiments, oligonucleotides are selective between a target and non-target, even though both target and non-target comprise the target sequence. In such embodiments, selectivity may result from relative accessibility of the target region of one nucleic acid molecule compared to the other.

In some embodiments, the antisense oligonucleotide may be at least 90% complementary over its entire length to a portion of the target RNA. In some embodiments, the antisense oligonucleotide may be at least 93% complementary over its entire length to a portion of the target RNA. In some embodiments, the antisense oligonucleotide may be at least 95% complementary over its entire length to a portion of the target RNA. In some embodiments, the antisense oligonucleotide may be at least 98% complementary over its entire length to a portion of the target RNA. In some embodiments, the antisense oligonucleotide may be at least 99% complementary over its entire length to a portion of the target RNA. In some embodiments, the antisense oligonucleotide may be 100% complementary over its entire length to a portion of the target RNA.

Certain embodiments provide a compound targeting a gene, wherein the compound comprises at least 8, at least 9, at least 10, at least 11, at least 12, at least 13, at least 14, at least 15, at least 16, at least 17, at least 18, at least 19, at least 20, at least 21, or 22 contiguous nucleobases complementary to an equal length portion of any target RNA. In some embodiments, the antisense oligonucleotide may comprise at least 12 contiguous nucleobases complementary to an equal length portion of the target RNA.

Certain embodiments provide compositions and methods comprising administering to an animal an antisense compound or composition disclosed herein. In certain embodiments, administering the antisense compound prevents, treats, ameliorates, or slows progression of disease or condition related to the expression of a gene or activity of a protein.

The invention further provides a method for inhibiting gene expression. The method comprising providing one or more antisense oligonucleotides described herein to a subject.

The invention further provides a method for treating a disease or disorder wherein inhibiting a gene expression would be beneficial. For example, a disease or disorder that results from an abnormal expression or product of a cellular gene. The method comprising providing one or more antisense oligonucleotides described herein to a subject.

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid is a pre-mRNA. In certain embodiments, an antisense oligonucleotide modulates splicing of a pre-mRNA.

In some embodiments, the invention provides a method for inhibiting gene expression comprising administering an antisense oligonucleotide complementary to a nucleotide sequence of a target RNA as disclosed herein.

In some embodiments, the antisense oligonucleotide comprises a 3′ domain, and a 5′ domain; wherein each nucleotide of the 3′ domain comprises a deoxyribonucleotide and a phosphodiester or phosphothioate internucleotide linkage or combinations thereof. In some embodiments, the hybridization of the antisense compound to a target nucleic acid results in cleavage at the 8^{th}, 9^{th} or 10^{th} position from the 3′ end. In some embodiments, the antisense oligonucleotide is administered locally.

In some embodiments, the antisense oligonucleotide comprises a 3′ domain, a 5′ domain and a 5′ end blocking agent; wherein each nucleotide of the 3′ domain comprises a natural nucleobase and a natural sugar. In some embodiments, the hybridization of the antisense compound to a target nucleic acid results in cleavage at the 8^{th}, 9^{th} or 10^{th} position from the 3′ end. In some embodiments, the 5′ blocking agent prevents a further cleavage. In some embodiments, the antisense oligonucleotide is administered locally.

In some embodiments, the invention provides a method inhibiting allele-specific gene expression. For example, the RNA target can be expressed from a first allele (e.g., a mutant allele), even when the first allelic mRNA differs from a second allele (e.g., wild-type allele) by only a single nucleotide, as is the case with certain mutations, for example, point mutations. The term “allele” refers to one of two alternate forms of a gene that can have the same locus on homologous chromosomes. Two different alleles may be responsible for alternative traits, e.g., one allele can be dominant over the other. The term “dominant allele” refers to an allele from which a trait is preferentially manifested as a phenotype. “Allele specific inhibition of expression” refers to the ability to significantly inhibit expression of one allele of a gene over another, e.g., when both alleles are present in the same cell. For example, the alleles can differ by one, two, or three or more nucleotides in the target region. In some embodiments, one allele is associated with disease causation, e.g., a disease correlated to a dominant gain-of-function mutation.

In some embodiments, the method of inhibiting allele-specific gene expression comprises administering an antisense oligonucleotide as disclosed herein, wherein the antisense oligonucleotide compound comprises a sequence complementary to a region of the target RNA encoding a point mutation and wherein the nucleotide within the antisense oligonucleotide complementary to the point mutation is located at the 9^{th} or 10^{th} nucleotide position from the 3′ end of the antisense oligonucleotide. Without wishing to be bound to any particular theory, the antisense oligonucleotide compounds of the present invention are capable of single nucleotide discrimination and selective down-regulation of expression of their target alleles. In some embodiments, the antisense oligonucleotide is administered locally.

The term “point mutation” refers to a single-base substitution observed in a target nucleotide sequence compared with the corresponding nucleotide sequence of a non-target sequence (e.g., a wild-type or normal allele). In this context, the “wild-type allele” refers to common naturally occurring alleles in the allele population of the same type of gene, wherein a protein encoded by this allele has normal function and/or activity. The point mutation may be any of congenitally occurring mutations and postnatally acquired mutations. Further point mutations include missense mutations that bring about amino acid substitution, silent mutations that do not result in amino acid substitution but causes change to a degenerate codon, a nonsense mutation that leads to the appearance of a stop codon, and a mutation at a splicing site. In certain embodiments, the point mutation is a dominant point mutation. A “dominant point mutation” refers to a point mutation that confers a dominant trait on the allele, or a dominant mutation-associated (or -linked) point mutation in one transcript.

The target allele may specify the amino acid sequence of a mutant protein associated with a pathological condition. For example, the protein may be a gain-of-function (e.g., a dominant gain-of-function) mutant protein. In a preferred aspect, the mutant protein is associated with a disease or disorder which is correlated with expression of a particular allele of a gene, e.g., a dominant gain-of-function mutation. The term “gain-of-function mutation” as used herein, refers to any mutation in a gene in which the protein encoded by said gene (i.e., the mutant protein) acquires a function not normally associated with the protein (i.e., the wild type protein) causes or contributes to a disease or disorder. The gain-of-function mutation can be a deletion, addition, or substitution of a nucleotide or nucleotides in the gene which gives rise to the change in the function of the encoded protein. In one embodiment, the gain-of-function mutation is a point mutation. In one embodiment, the gain-of-function mutation is a translocation.

In one embodiment, the gain-of-function mutation changes the function of the mutant protein or causes interactions with other proteins. In another embodiment, the gain-of-function mutation causes a decrease in or removal of normal wild-type protein, for example, by interaction of the altered, mutant protein with said normal, wild-type protein. The trait in which the dominant point mutation is involved is not particularly limited and is preferably a trait to be suppressed. Examples thereof include a mutation involved in the onset of a disease and a mutation involved in abnormal morphology. Gain-of-function disorders are a class of disease or disorders characterized by a gain-of-function mutation. For example, such disorders may include amyotrophic lateral sclerosis, Huntington's disease, Alzheimer's disease, Parkinson's disease, as well as cancer.

Other examples of gain-of-function mutations include the KIT receptor, which has been linked to a number of gastrointestinal stromal tumors. Naturally occurring mutations in G protein alpha subunits and in G protein-coupled receptors have been linked to a number of human diseases, including endocrine disorders. Germline loss of function mutations in the ubiquitously expressed Gs-alpha gene have been identified as the cause of generalized hormone resistance and dysmorphic features in the inherited disorder pseudohypoparathyroidism type 1a. Somatic gain-of-function mutations in Gs-alpha have been identified as the cause of the McCune-Albright syndrome, a sporadic disorder in which affected individuals have varying combinations of endocrine hyperfunction, cafe-au-lait skin pigmentation, and polyostotic fibrous dysplasia. These mutant genes and conditions may be targeted with 3GA compounds in accordance with the invention.

Further, gain-of-function mutations in the thyrotropin receptor (TSHR, a G-protein coupled receptor) are correlated with toxic follicular thyroid adenoma, a condition caused by excessive quantities of thyroid hormones. Gain-of-function mutations in TSH receptor genes have also been linked to hereditary toxic thyroid hyperplasia, another condition caused by excessive quantities of thyroid hormones. Mutations of the superoxide dismutase (SOD) gene have been linked to certain familial forms of ALS. Mutations in protein-tyrosine phosphatase, nonreceptor-type 11 (PTPN11) have been correlated with Noonan syndrome, an autosomal dominant disorder characterized by dysmorphic facial features, proportionate short stature and heart disease. Hereditary pancreatitis is associated with mutations in human cationic trypsinogen. Brachydactyly type B (BDB), an autosomal dominant disorder characterized by terminal deficiency of the fingers and toes, is believed to be associated with dominant gain-of-function mutation in ROR2, which encodes an orphan receptor tyrosine kinase, von Willebrand disease, particularly Type 2A and 2B, is another disease which may be associated with a dominant gain-of-function mutation. A dominant gain-of-function mutation has been described in p53 that results in oncogenic activation of that gene. In addition, Creutzfeldt-Jakob disease has been associated with a dominant gain-of-function mutation in the prion protein gene, the PRNP E200K mutation. Testotoxicosis is an autosomal dominant condition caused by a gain-of-function mutation in the LH receptor.

In some embodiments, the target RNA encodes an oncogene, such as BRAF, or a Ras protein such as H-Ras, K-Ras, or N-Ras. These oncogenes contain point mutations responsible for their tumorigenic activity in cells. For example, the antisense oligonucleotide compound may comprise oligonucleotides that are complementary to a T>A mutation at position 9, 10, 11 of the oligonucleotides (numbered from the 3′ end). In some embodiments, the antisense oligonucleotide compound may comprise oligonucleotides that are complementary to a T>A mutation at position 11 of the oligonucleotides (numbered from the 3′ end).

In some embodiments, the target RNA and non-target encode an enzyme, where a point mutation results in stronger activity (or abnormal activity) of the encoded enzyme, as compared to the enzyme encoded by the non-target RNA. For example, the target and non-target RNA may encode a kinase. In various embodiments, the enzyme is an oxidoreductase, transferase, hydrolase, lyase, isomerase, or ligase. In some embodiments, the enzyme is a superoxide dismutase or a triglyceride hydrolase. In still other embodiments, the target RNA and non-target RNA encode a transcriptional activator, such as MYD88. The MYD88 L265P variant is the most prevalent mutation in patients with Waldenstrom's macroglobulinemia (WM), a type of non-Hodgkin's lymphoma. MYD88 L265P often results from a T→C transversion. Signaling studies showed that the mutant protein that is encoded by MYD88 L265P triggers tumor growth through the activation of nuclear factor kappa light-chain enhancer of activated B cells (NF-κB) by Bruton's tyrosine kinase. (Treon et al., MYD88 Mutations and Response to Ibrutinib in Waldenstrom's Macroglobulinemia, N Engl J Med 2015; 373, 584-586 (2015)).

In certain embodiments, the expressed gene product of a mutant allele results in aggregation of the mutant proteins causing disease. In certain embodiments, the expressed gene product of a mutant allele results in gain of function causing disease. In certain embodiments, genes with an autosomal dominant mutation resulting in a toxic gain of function of the protein are the APP gene encoding amyloid precursor protein involved in Alzheimer's disease (Gene, 371: 68, 2006); the PrP gene encoding prion protein involved in Creutzfeldt-Jakob disease and in fatal familial insomnia (Nat. Med. 1997, 3: 1009); GFAP gene encoding glial fibrillary acidic protein involved in Alexander disease (J. Neurosci. 2006, 26:111623); alpha-synuclein gene encoding alpha-synuclein protein involved in Parkinson's disease (J. Clin. Invest. 2003, 111: 145); SOD-1 gene encoding the SOD-1 protein involved in amyotrophic lateral sclerosis (Science 1998, 281: 1851); atrophin-1 gene encoding atrophin-1 protein involved in dentato-rubral and pallido-luysian atrophy (DRPA) (Trends Mol. Med. 2001, 7: 479); SCA1 gene encoding ataxin-1 protein involved in spino-cerebellar ataxia-1 (SCA1) (Protein Sci. 2003, 12: 953); PLP gene encoding proteolipid protein involved in Pelizaeus-Merzbacher disease (NeuroMol. Med. 2007, 4: 73); DYT1 gene encoding torsinA protein involved in Torsion dystonia (Brain Res. 2000, 877: 379); and alpha-B crystalline gene encoding alpha-B crystalline protein involved in protein aggregation diseases, including cardiomyopathy (Cell 2007, 130: 427); alpha1-antitrypsin gene encoding alpha1-antitrypsin protein involved in chronic obstructive pulmonary disease (COPD), liver disease and hepatocellular carcinoma (New Engl J. Med. 2002, 346: 45); Ltk gene encoding leukocyte tyrosine kinase protein involved in systemic lupus erythematosus (Hum. Mol. Gen. 2004, 13: 171); PCSK9 gene encoding PCSK9 protein involved in hypercholesterolemia (Hum Mutat. 2009, 30: 520); prolactin receptor gene encoding prolactin receptor protein involved in breast tumors (Proc. Natl. Assoc. Sci. 2008, 105: 4533); CCLS gene encoding the chemokine CCLS involved in COPD and asthma (Eur. Respir. J. 2008, 32: 327); PTPN22 gene encoding PTPN22 protein involved in Type 1 diabetes, Rheumatoid arthritis, Graves disease, and SLE (Proc. Natl. Assoc. Sci. 2007, 104: 19767); androgen receptor gene encoding the androgen receptor protein involved in spinal and bulbar muscular atrophy or Kennedy's disease (J Steroid Biochem. Mol. Biol. 2008, 108: 245); CHMP4B gene encoding chromatin modifying protein-4B involved in progressive childhood posterior subcapsular cataracts (Am. J. Hum. Genet. 2007, 81: 596); FXR/NR1H4 gene encoding Farnesoid X receptor protein involved in cholesterol gallstone disease, arthrosclerosis and diabetes (Mol. Endocrinol. 2007, 21: 1769); ABCA1 gene encoding ABCA1 protein involved in cardiovascular disease (Transl. Res. 2007, 149: 205); CaSR gene encoding the calcium sensing receptor protein involved in primary hypercalciuria (Kidney Int. 2007, 71: 1155); alpha-globin gene encoding alpha-globin protein involved in alpha-thalassemia (Science 2006, 312: 1215); httlpr gene encoding HTTLPR protein involved in obsessive compulsive disorder (Am. J. Hum. Genet. 2006, 78: 815); AVP gene encoding arginine vasopressin protein in stress-related disorders such as anxiety disorders and comorbid depression (CNS Neurol. Disord. Drug Targets 2006, 5: 167); GNAS gene encoding G proteins involved in congenital visual defects, hypertension, metabolic syndrome (Trends Pharmacol. Sci. 2006, 27: 260); APAF1 gene encoding APAF1 protein involved in a predisposition to major depression (Mol. Psychiatry. 2006, 11: 76); TGF-beta1 gene encoding TGF-beta1 protein involved in breast cancer and prostate cancer (Cancer Epidemiol. Biomarkers Prev. 2004, 13: 759); AChR gene encoding acetylcholine receptor involved in congenital myasthenic syndrome (Neurology 2004, 62: 1090); P2Y12 gene encoding adenosine diphosphate (ADP) receptor protein involved in risk of peripheral arterial disease (Circulation 2003, 108: 2971); LQT1 gene encoding LQT1 protein involved in atrial fibrillation (Cardiology 2003, 100: 109); RET protooncogene encoding RET protein involved in sporadic pheochromocytoma (J. Clin. Endocrinol. Metab. 2003, 88: 4911); filamin A gene encoding filamin A protein involved in various congenital malformations (Nat. Genet. 2003, 33: 487); TARDBP gene encoding TDP-43 protein involved in amyotrophic lateral sclerosis (Hum. Mol. Genet. 2010, 19: 671); SCA3 gene encoding ataxin-3 protein involved in Machado-Joseph disease (PLoS One 2008, 3: e3341); SCAT gene encoding ataxin-7 protein involved in spino-cerebellar ataxia-7 (PLoS One 2009, 4: e7232); and HTT gene encoding huntingtin protein involved in Huntington's disease (Neurobiol Dis. 1996, 3:183); and the CA4 gene encoding carbonic anhydrase 4 protein, CRX gene encoding cone-rod homeobox transcription factor protein, FSCN2 gene encoding retinal fascin homolog 2 protein, IMPDH1 gene encoding inosine monophosphate dehydrogenase 1 protein, NR2E3 gene encoding nuclear receptor subfamily 2 group E3 protein, NRL gene encoding neural retina leucine zipper protein, PRPF3 (RP18) gene encoding pre-mRNA splicing factor 3 protein, PRPF8 (RP13) gene encoding pre-mRNA splicing factor 8 protein, PRPF31 (RP11) gene encoding pre-mRNA splicing factor 31 protein, RDS gene encoding peripherin 2 protein, ROM1 gene encoding rod outer membrane protein 1 protein, RHO gene encoding rhodopsin protein, RP1 gene encoding RP1 protein, RPGR gene encoding retinitis pigmentosa GTPase regulator protein, all of which are involved in Autosomal Dominant Retinitis Pigmentosa disease (Adv Exp Med. Biol. 2008, 613:203).

In certain embodiments, the mutant allele is associated with any disease from the group consisting of Alzheimer's disease, Creutzfeldt-Jakob disease, fatal familial insomnia, Alexander disease, Parkinson's disease, amyotrophic lateral sclerosis, dentato-rubral and pallido-luysian atrophy DRPA, spino-cerebellar ataxia, Torsion dystonia, cardiomyopathy, chronic obstructive pulmonary disease (COPD), liver disease, hepatocellular carcinoma, systemic lupus erythematosus, hypercholesterolemia, breast cancer, asthma, Type 1 diabetes, Rheumatoid arthritis, Graves disease, SLE, spinal and bulbar muscular atrophy, Kennedy's disease, progressive childhood posterior subcapsular cataracts, cholesterol gallstone disease, arthrosclerosis, cardiovascular disease, primary hypercalciuria, alpha-thallasemia, obsessive compulsive disorder, Anxiety, comorbid depression, congenital visual defects, hypertension, metabolic syndrome, prostate cancer, congenital myasthenic syndrome, peripheral arterial disease, atrial fibrillation, sporadic pheochromocytoma, congenital malformations, Machado-Joseph disease, Huntington's disease, and Autosomal Dominant Retinitis Pigmentosa disease.

In some embodiments, the invention provides a method modulating splicing. In some embodiments, modulating gene splicing increases expression of a target protein or a target functional RNA. In some embodiments, the target RNA comprises a retained intron. In some embodiments the retained intron is flanked on one or both sides by an exon. In some embodiments, an exon flanks the 5′ splice site of the retained intron. In some embodiments, an exon flanks the 3′ splice site of the retained intron. In some embodiments, an exon flanks the 5′ splice site of the retained intron and an exon flanks the 3′ splice site of the retained intron. In some embodiments, the retained intron is constitutively spliced from the target RNA; thereby increasing a level of mRNA encoding the target protein or the target functional RNA and increasing expression of the target protein or the target functional RNA.

In some embodiments, the method of modulating splicing is useful to treat a subject having a condition caused by a deficient amount or activity of the target protein or a deficient amount or activity of the target functional RNA; and wherein the deficient amount or activity of the target protein or the target functional RNA is caused by haploinsufficiency of the target protein or the target functional RNA.

In some embodiments, the method of modulating splicing comprises administering an antisense oligonucleotide complementary as disclosed herein, wherein the antisense oligonucleotide compound comprises a sequence complementary to a region of the target RNA comprising a retained intron and wherein the nucleotides at the 8^{th} and/or 9^{th} positions from the 3 end of the antisense oligonucleotide are modified or are not complementary to the target RNA (i.e., mismatch). Without wishing to be bound to any particular theory, mismatch and/or modification of the nucleotides at the 9^{th} and/or 10^{th} positions from the 3 end of the antisense oligonucleotide allows the antisense oligonucleotide to bind the target RNA; however, the antisense oligonucleotide becomes RNase H inactive. In other words, the antisense oligonucleotide and target RNA will not be cleaved by RNase H. In some embodiments, the antisense oligonucleotide is administered locally.

The antisense oligonucleotides of the invention may be administered alone or in combination with any other agent or therapy. Agents or therapies can be co-administered or administered concomitantly. Such agent or therapy may be useful for treating or preventing the disease or condition and does not diminish the gene expression modulation effect of the antisense oligonucleotide according to the invention. Agent(s) useful for treating or preventing the disease or condition includes, but is not limited to, vaccines, antigens, antibodies, preferably monoclonal antibodies, cytotoxic agents, kinase inhibitors, allergens, antibiotics, siRNA molecules, antisense oligonucleotides, TLR antagonist (e.g., antagonists of TLR3 and/or TLR7 and/or antagonists of TLR8 and/or antagonists of TLR9), chemotherapeutic agents (both traditional chemotherapy and modern targeted therapies), targeted therapeutic agents, activated cells, peptides, proteins, gene therapy vectors, peptide vaccines, protein vaccines, DNA vaccines, adjuvants, and co-stimulatory molecules (e.g., cytokines, chemokines, protein ligands, trans-activating factors, peptides or peptides comprising modified amino acids), or combinations thereof. Alternatively, the antisense oligonucleotides according to the invention can be administered in combination with other compounds (for example lipids or liposomes) to enhance the specificity or magnitude of the gene expression modulation of the antisense oligonucleotides according to the invention.

The antisense oligonucleotides of the invention may be administered can be by any suitable route, including, without limitation, parenteral, mucosal delivery, oral, sublingual, transdermal, topical, inhalation, intratumoral, intravenous, subcutaneous, intrathecal, intranasal, aerosol, intraocular, intratracheal, intrarectal, vaginal, by gene gun, dermal patch or in eye drop or mouthwash form. In any of the methods according to the invention, administration of antisense oligonucleotides according to the invention, alone or in combination with any other agent, can be directly to a tissue or organ such as, but not limited to, the bladder, liver, lung, kidney or lung. In certain embodiments, administration of antisense oligonucleotides according to the invention, alone or in combination with any other agent, is by intramuscular administration. In certain embodiments, administration of antisense oligonucleotides according to the invention, alone or in combination with any other agent, is by mucosal administration. In certain embodiments, administration of antisense oligonucleotides according to the invention, alone or in combination with any other agent, is by oral administration. In certain embodiments, administration of antisense oligonucleotides according to the invention, alone or in combination with any other agent, is by intrarectal administration. In certain embodiments, administration of antisense oligonucleotides according to the invention, alone or in combination with any other agent, is by intrathecal administration. In certain embodiments, administration of antisense oligonucleotides according to the invention, alone or in combination with any other agent, is by intratumoral administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Administration of the antisense oligonucleotides according to the invention can be carried out using known procedures using an effective amount and for periods of time effective to reduce symptoms or surrogate markers of the disease. For example, an effective amount of an antisense oligonucleotide according to the invention for treating a disease and/or disorder could be that amount necessary to alleviate or reduce the symptoms, or delay or ameliorate a tumor, cancer, or bacterial, viral or fungal infection. In the context of administering a composition that modulates gene expression, an effective amount of an antisense oligonucleotide according to the invention is an amount sufficient to achieve the desired modulation as compared to the gene expression in the absence of the antisense oligonucleotide according to the invention. The effective amount for any particular application can vary depending on such factors as the disease or condition being treated, the particular oligonucleotide being administered, the size of the subject, or the severity of the disease or condition. One of ordinary skill in the art can empirically determine the effective amount of a particular antisense oligonucleotide without necessitating undue experimentation.

When administered systemically, the therapeutic composition is preferably administered at a sufficient dosage to attain a blood level of compound according to the invention from about 0.0001 micromolar to about 10 micromolar. For localized administration, much lower concentrations than this may be effective, and much higher concentrations may be tolerated. Preferably, a total dosage of compound according to the invention ranges from about 0.001 mg per patient per day to about 200 mg per kg body weight per day. In certain embodiments, the total dosage may be 0.08, 0.16, 0.32, 0.48, 0.32, 0.64, 1, 10 or 30 mg/kg body weight administered daily, twice weekly or weekly. It may be desirable to administer simultaneously, or sequentially a therapeutically effective amount of one or more of the therapeutic compositions of the invention to an individual as a single treatment episode.

The methods according to this aspect of the invention are useful for model studies of gene expression. The methods are also useful for the prophylactic or therapeutic treatment of human or animal disease. For example, the methods are useful for pediatric and veterinary inhibition of gene expression applications.

Certain embodiments provide a kit for treating, preventing, or ameliorating a disease, disorder or condition as described herein wherein the kit comprises: (i) an antisense oligonucleotide as described herein; and optionally (ii) a second agent or therapy as described herein. A kit of the present invention can further include instructions for using the kit to treat, prevent, or ameliorate a disease, disorder or condition as described herein.

### Cell Culture and Antisense Compounds Treatment

The effects of antisense compounds on the level, activity or expression of target nucleic acids can be tested in vitro in a variety of cell types. Cell types used for such analyses are available from commercial vendors (e.g., American Type Culture Collection, Manassas, Va.; Zen-Bio, Inc., Research Triangle Park, N.C.; Clonetics Corporation, Walkersville, Md.) and are cultured according to the vendor's instructions using commercially available reagents (e.g., Invitrogen Life Technologies, Carlsbad, Calif.). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

Cells may be treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, Calif.). Antisense oligonucleotides may be mixed with LIPOFECTIN in OPTI-MEM 1 (Invitrogen, Carlsbad, Calif.) to achieve the desired final concentration of antisense oligonucleotide and a LIPOFECTIN concentration that may range from 2 to 12 ug/mL per 100 nM antisense oligonucleotide.

Another reagent used to introduce antisense oligonucleotides into cultured cells includes LIPOFECTAMINE (Invitrogen, Carlsbad, Calif.). Antisense oligonucleotide is mixed with LIPOFECTAMINE in OPTI-MEM 1 reduced serum medium (Invitrogen, Carlsbad, Calif.) to achieve the desired concentration of antisense oligonucleotide and a LIPOFECTAMINE concentration that may range from 2 to 12 ug/mL per 100 nM antisense oligonucleotide.

Another technique used to introduce antisense oligonucleotides into cultured cells includes electroporation.

Cells are treated with antisense oligonucleotides by routine methods. Cells may be harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+mRNA. Methods of RNA isolation are well known in the art. RNA is prepared using methods well known in the art, for example, using the TRIZOL Reagent (Invitrogen, Carlsbad, Calif.) according to the manufacturer's recommended protocols.

### Analysis of Inhibition of Target Levels or Expression

Inhibition of levels or expression of a target nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents may be obtained from Invitrogen (Carlsbad, Calif.). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, Calif.). Cyclophilin A expression is quantified by real time PCR, by being run simultaneously with the target, multiplexing, or separately. Total RNA is quantified using RIBOGREEN RNA quantification reagent (Invetrogen, Inc. Eugene, Oreg.). Methods of RNA quantification by RIBOGREEN are taught in Jones, L. J., et al., (Analytical Biochemistry, 1998, 265, 368-374). A CYTOFLUOR 4000 instrument (PE Applied Biosystems) is used to measure RIBOGREEN fluorescence.

Probes and primers are designed to hybridize to a target nucleic acid. Methods for designing real-time PCR probes and primers are well known in the art, and may include the use of software such as PRIMER EXPRESS Software (Applied Biosystems, Foster City, Calif.).

### Analysis of Protein Levels

Antisense inhibition of target nucleic acids can be assessed by measuring corresponding protein levels. Protein levels of can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of a target nucleic acid and produce phenotypic changes.

Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated and changes in nucleic acid expression are measured.

### Certain Indications

In certain embodiments, provided herein are methods of treating an individual comprising administering one or more pharmaceutical compositions described herein. Certain embodiments include treating an individual in need thereof by administering to an individual a therapeutically effective amount of an antisense compound described herein.

In one embodiment, administration of a therapeutically effective amount of an antisense compound targeted to a nucleic acid is accompanied by monitoring of the corresponding target levels in an individual, to determine an individual's response to administration of the antisense compound. An individual's response to administration of the antisense compound may be used by a physician to determine the amount and duration of therapeutic intervention.

In certain embodiments, administration of an antisense compound targeted to a nucleic acid results in reduction of expression by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

### Examples

### Synthesis of Antisense Oligonucleotides

Antisense oligonucleotides according to the invention can be synthesized by procedures that are well known in the art, such as phosphoramidate or H-phosphonate chemistry which can be carried out manually or by an automated synthesizer. For example, the antisense oligonucleotides of the invention may be synthesized by a linear synthesis approach.

ARNA compounds employed in the study have been synthesized using phosphoramidite chemistry. These protocols are described in detail, for example in https://pubs.rsc.org/en/content/chapter/bk9781788012096-00453/978-1-78801-209-6, which is incorporated herein by reference.

### Inhibition of Target RNA by Antisense Oligonucleotide

Antisense oligonucleotides were designed targeting a PCSK9 nucleic acid and were tested for their effects on PCSK9 mRNA in vitro. The antisense oligonucleotides were tested in a series of experiments that had similar culture conditions. The results for each experiment are presented in separate tables shown below.

Hepa 1-6 cells were cultured in DMEM medium plus 10% FBS and 100 U/ml Pen/Strep (cells from ATCC). The cells were seeded and allowed to incubate overnight so that they are ˜70% confluent at the time of transfection—˜100,000 cells/ml/12 well plate. Cell media was changed and 900 μl was added to each well. The antisense compounds were mixed with Lipofectamine in Opti-MEM medium, added to lipid (1:1 ratio) and incubated for 15-20 minutes. 100 μl was added to each well for an antisense concentration of 100 nM. After a treatment period of about 16 to 48 hours, cells were harvested for RNA and/or protein analysis.

Culture supernatants were assayed for AK release cytotoxicity assay. Taqman probes for mPCSK9 and PPIB or HPRT1 (housekeeping controls) were used (probes provided by ThermoFisher).

| | | Knockdown of PC SK9(%) | |
|---|---|---|---|
| Compound | Sequence | 100 nM | 25 nM |
| 1 | 5′-TCTCCTCCATCAGCACC-3′ | 43 | 14 |
| 7 | 5′-TCTCCTCCCTCAGCACC-3′ | 20 | 13 |
| 8 | 5′-TCTCCTCAATCAGCACC-3′ | 16 | 11 |
| 3 | 5′-UCUCCTCCATCAGCACC-3′ | 31 | |
| 5 | 5′-UCUCCTCCCTCAGCACC-3′ | 23 | |
| 4 | 5′-UCTCCUCCATCAGCACC-3′ | 53 | 32 |
| 6 | 5′-UCTCCUCCCTCAGCACC-3′ | 20 | 7 |
| 11 | 5′-UCTCCUCAATCAGCACC-3′ | 18 | 11 |
| 14 | 5′-UcTcCuCCATCAGCACC-3′ | 60 | 43 |
| 15 | 5′-UcTcCuCCCTCAGCACC-3′ | 39 | |
| 9 | 5′-UCUCCTCCATCAGCACC-3′ | 27 | 13 |
| 10 | 5′-UCUCCTCCAUCAGCACC-3′ | 34 | 23 |
| 12 | 5′-UCTCCUCCATCAGCACC-3′ | 63 | 36 |
| 13 | 5′-UCTCCTCCATCAGCACC-3′ | 68 | 45 |

7 and 8 have a mismatch at the 9^{th} and 10^{th} positions from the 3′ end, respectively, which resulted in a reduction of activity.

Introducing modifications in the 5′ domain as in 4 and 14 increased the potency and specificity of the antisense oligonucleotide. Mismatches at the 9^{th} and 10^{th} positions from the 3′ end as in 5, 6, 11 and 15 resulted in a reduction of activity and specificity.

While this invention has been particularly shown and described with references to preferred embodiments thereof, it will be understood by those skilled in the art that various changes in form and details may be made therein without departing from the scope of the invention encompassed by the appended claims.

1. An antisense oligonucleotide compound 17 to 25 nucleotides in length comprising at least 12 contiguous nucleobases complementary to an equal length portion of a target RNA sequence, wherein the antisense oligonucleotide compound comprises a 3′ domain and a 5′ domain, which is contiguous with the 3′ domain,
wherein the 3′ domain is 10 to 12 nucleotides in length and each nucleotide comprises a deoxyribonucleotide and a phosphodiester or phosphothioate internucleotide linkage or combinations thereof; and
wherein the 5′ domain is 5 to 15 nucleotides in length, and wherein the 5′ domain comprises unmodified deoxyribonucleotides, unmodified ribonucleotides, modified deoxyribonucleotides, modified ribonucleotides, or combinations thereof, provided that the 5′ domain comprises at least 1 modified deoxyribonucleotide or modified ribonucleotide comprising a modified sugar and/or backbone.

2. The antisense oligonucleotide according to claim 1, wherein the 3′ domain is 12 nucleotides in length and comprises nucleotides at positions 1, 2, 3, 4, 5, 6, 7, 8, 9, 10, 11, and 12 from the 3′ end.

3. The antisense oligonucleotide according to claim 1, wherein the 3′ domain is 11 nucleotides in length and comprises nucleotides at positions 1, 2, 3, 4, 5, 6, 7, 8, 9, 10, and 11 from the 3′ end.

4. The antisense oligonucleotide according to claim 1, wherein the 3′ domain is 10 nucleotides in length and comprises nucleotides at positions 1, 2, 3, 4, 5, 6, 7, 8, 9, and 10 from the 3′ end.

5. The antisense oligonucleotide according to claim 1, wherein the nucleotides of the 3′ domain comprise a natural nucleobase.

6. The antisense oligonucleotide according to claim 1, wherein at least one of the nucleotides of the 3′ domain comprises a modified nucleobase.

7. The antisense oligonucleotide according to claim 6, wherein the nucleotides at the 9^{th} or 10^{th} positions are not modified.

8. The antisense oligonucleotide according to claim 1, wherein the nucleotide at the 11^{th} position is not modified.

9. The antisense oligonucleotide according to claim 1, wherein the oligonucleotide comprises at least one phosphorothioate internucleotide linkage.

10. The antisense oligonucleotide according to claim 9, wherein at least half of the internucleotide linkages are phosphorothioate.

11. The antisense oligonucleotide according to claim 1, wherein the antisense oligonucleotide is single stranded.

12. The antisense oligonucleotide according to claim 1, wherein the 5′ domain comprises at least 1 modified deoxyribonucleotide or modified ribonucleotide comprising a modified sugar.

13. The antisense oligonucleotide according to claim 1, wherein the 5′ domain comprises at least 1 modified deoxyribonucleotide or modified ribonucleotide comprising a nonphosphorus-based backbone.

14. The antisense oligonucleotide according to claim 1, wherein at least three of the nucleotides of the 5′ domain are modified with a deoxyribonucleotide or modified ribonucleotide comprising a modified sugar and/or backbone.

15. The antisense oligonucleotide according to claim 1, further comprising a 5′ blocking agent.

16. The antisense oligonucleotide according to claim 1, wherein the antisense oligonucleotide is at least 90% complementary over its entire length to a portion of the target RNA.

17. The antisense oligonucleotide according to claim 1, wherein the target RNA may be an mRNA, pre-mRNA. ncRNA, or microRNA.

18. The antisense oligonucleotide according to claim 17, wherein the target RNA is mRNA.

19. A pharmaceutical composition comprising the antisense oligonucleotide according to claim 1 and a pharmaceutically acceptable carrier.

20. A method for inhibiting gene expression comprising administering an antisense oligonucleotide according to claim 1 or a composition according to claim 19, wherein the antisense oligonucleotide is complementary to a nucleotide sequence of a target RNA.

21. A method for inhibiting allele-specific gene expression comprising administering an antisense oligonucleotide according to claim 1 or a composition according to claim 19, wherein the antisense oligonucleotide is complementary to a nucleotide sequence of a target allele RNA.

22. The method according to claim 21, wherein the target allele comprises a point mutation.

23. The method according to claim 22, wherein the antisense oligonucleotide compound comprises a sequence complementary to a region of the target allele RNA encoding a point mutation and wherein the nucleotide within the antisense oligonucleotide complementary to the point mutation is located at the 9^{th}, 10^{th} or 11^{th} nucleotide position from the 3′ end of the antisense oligonucleotide.

24. The method according to claim 23, wherein the antisense oligonucleotide compound comprises a sequence complementary to a region of the target allele RNA encoding a point mutation and wherein the nucleotide within the antisense oligonucleotide complementary to the point mutation is located at the 9^{th} or 10^{th} nucleotide position from the 3′ end of the antisense oligonucleotide.

25. (canceled)

26. The method according to claim 20, wherein in the method is useful for treating a subject having disease or disorder wherein inhibiting expression of a gene would be beneficial.

27. The method according to claim 26, wherein the disease or disorder results from abnormal expression or product of a cellular gene.

28. The method according to claim 20, wherein the antisense oligonucleotide is administered locally.

29. The method according to claim 21, wherein in the method is useful for treating a subject having disease or disorder wherein inhibiting expression of a gene would be beneficial.

30. The method according to claim 29, wherein the disease or disorder results from abnormal expression or product of a cellular gene.

31. The method according to claim 21, wherein the antisense oligonucleotide is administered locally.


SEQUENCE LISTING (17 sequences)

SEQ ID NO: 1 - DNA, 17 bp, Artificial Sequence
  Synthetic oligonucleotide
tctcctccatcagcacc

SEQ ID NO: 2 - DNA, 19 bp, Artificial Sequence
  Synthetic oligonucleotide
ggtctcctccatcagcacc

SEQ ID NO: 3 - DNA, 17 bp, Artificial Sequence
  Synthetic oligonucleotide
  misc_feature (1)..(1): 2′-O-methyl phosphorothioate
  misc_feature (3)..(3): 2′-O-methyl phosphorothioate
  misc_feature (5)..(5): 2′-O-methyl phosphorothioate
  misc_feature (7)..(8): 2′-O-methyl phosphorothioate
ucucctccatcagcacc

SEQ ID NO: 4 - DNA, 17 bp, Artificial Sequence
  Synthetic oligonucleotide
  misc_feature (1)..(2): 2′-O-methyl phosphorothioate
  misc_feature (4)..(4): 2′-O-methyl phosphorothioate
  misc_feature (6)..(7): 2′-O-methyl phosphorothioate
uctccuccatcagcacc

SEQ ID NO: 5 - DNA, 17 bp, Artificial Sequence
  Synthetic oligonucleotide
  misc_feature (1)..(1): 2′-O-methyl phosphorothioate
  misc_feature (3)..(3): 2′-O-methyl phosphorothioate
  misc_feature (5)..(5): 2′-O-methyl phosphorothioate
  misc_feature (7)..(8): 2′-O-methyl phosphorothioate
ucucctccctcagcacc

SEQ ID NO: 6 - DNA, 17 bp, Artificial Sequence
  Synthetic oligonucleotide
  misc_feature (1)..(2): 2′-O-methyl phosphorothioate
  misc_feature (4)..(4): 2′-O-methyl phosphorothioate
  misc_feature (6)..(7): 2′-O-methyl phosphorothioate
uctccuccctcagcacc

SEQ ID NO: 7 - DNA, 17 bp, Artificial Sequence
  Synthetic oligonucleotide
tctcctccctcagcacc

SEQ ID NO: 8 - DNA, 17 bp, Artificial Sequence
  Synthetic oligonucleotide
tctcctcaatcagcacc

SEQ ID NO: 9 - DNA, 17 bp, Artificial Sequence
  Synthetic oligonucleotide
  misc_feature (1)..(1): 2′-O-methyl phosphorothioate
  misc_feature (3)..(3): 2′-O-methyl phosphorothioate
  misc_feature (5)..(5): 2′-O-methyl phosphorothioate
  misc_feature (7)..(7): 2′-O-methyl phosphorothioate
  misc_feature (9)..(10): 2′-O-methyl phosphorothioate
ucucctccaucagcacc

SEQ ID NO: 10 - DNA, 17 bp, Artificial Sequence
  Synthetic oligonucleotide
  misc_feature (1)..(1): 2′-O-methyl phosphorothioate
  misc_feature (3)..(3): 2′-O-methyl phosphorothioate
  misc_feature (5)..(5): 2′-O-methyl phosphorothioate
  misc_feature (7)..(7): 2′-O-methyl phosphorothioate
  misc_feature (9)..(9): 2′-O-methyl phosphorothioate
  misc_feature (11)..(12): 2′-O-methyl phosphorothioate
ucucctccatcagcacc

SEQ ID NO: 11 - DNA, 17 bp, Artificial Sequence
  Synthetic oligonucleotide
  misc_feature (1)..(2): 2′-O-methyl phosphorothioate
  misc_feature (4)..(4): 2′-O-methyl phosphorothioate
  misc_feature (6)..(7): 2′-O-methyl phosphorothioate
uctccucaatcagcacc

SEQ ID NO: 12 - DNA, 17 bp, Artificial Sequence
  Synthetic oligonucleotide
  misc_feature (1)..(2): 2′-O-methyl phosphorothioate
  misc_feature (4)..(4): 2′-O-methyl phosphorothioate
  misc_feature (6)..(6): 2′-O-methyl phosphorothioate
uctccuccatcagcacc

SEQ ID NO: 13 - DNA, 17 bp, Artificial Sequence
  Synthetic oligonucleotide
  misc_feature (1)..(2): 2′-O-methyl phosphorothioate
  misc_feature (4)..(4): 2′-O-methyl phosphorothioate
uctcctccatcagcacc

SEQ ID NO: 14 - DNA, 17 bp, Artificial Sequence
  Synthetic oligonucleotide
  misc_feature (1)..(1): 2′-O-methyl phosphorothioate
  misc_feature (2)..(2): 2′-O-methyl phosphodiester
  misc_feature (4)..(4): 2′-O-methyl phosphodiester
  misc_feature (6)..(6): 2′-O-methyl phosphodiester
  misc_feature (7)..(7): 2′-O-methyl phosphorothioate
uctccuccatcagcacc

SEQ ID NO: 15 - DNA, 17 bp, Artificial Sequence
  Synthetic oligonucleotide
  misc_feature (1)..(1): 2′-O-methyl phosphorothioate
  misc_feature (2)..(2): 2′-O-methyl phosphodiester
  misc_feature (4)..(4): 2′-O-methyl phosphodiester
  misc_feature (6)..(6): 2′-O-methyl phosphodiester
  misc_feature (7)..(7): 2′-O-methyl phosphorothioate
uctccuccctcagcacc

SEQ ID NO: 16 - DNA, 3512 bp, Mus musculus
tggggattaagaggggggaatgtaacaggtcccgtttgcagcccaattaggatttggggttttgtcctcactctgagcgtcatttgacgctgtctggggagggcgaggccgaaacctgatcctttagtaccggggcccgttaatgtttaatcagagaggatcttccgatggggctcggggtggcgtgatctcccggcccccaggcgtccagtacccacaccccagaaggcttccaccttcacgtggacgcgcaggctgccggtgggctcccgttctctctctctttctgaggctagaggactgagccagtccttggctccccagagacatcacggcccgcagccccggagccaagtgccccgagtcccaggcgtccatgtccttcccgaggccgcgcgcacctctcctcgccccgatgggcacccactgctctgcgtggctgcggtggccgctgttgccgctgttgccgccgctgctgctgctgttgctgctactgtgccccaccggcgctggtgcccaggacgaggatggagattatgaagagctgatgctcgccctcccgtcccaggaggatggcctggctgatgaggccgcacatgtggccaccgccaccttccgccgttgctccaaggaggcctggaggctgccaggaacctacattgtggtgctgatggaggagacccagaggctacagattgaacaaactgcccaccgcctgcagacccgggctgcccgccggggctatgtcatcaaggttctacatatcttttatgacctcttccctggcttcttggtgaagatgagcagtgacctgttgggcctggccctgaagttgccccatgtggagtacattgaggaagactcctttgtcttcgcccagagcatcccatggaacctggagcgaattatcccagcatggcaccagacagaggaagaccgctcccctgatggaagcagccaggtggaggtgtatctcttagataccagcatccagggtgcccatcgggagattgagggcagggtcaccatcaccgacttcaacagcgtgccggaggaggatgggacacgcttccacagacaggcgagcaagtgtgacagccacggcacccacctggcaggtgtggtcagcggccgggatgctggtgtggccaagggcaccagcctgcacagcctgcgtgtgctcaactgtcaagggaagggcacagtcagcggcaccctcataggcctggagtttattcggaagagtcagctaatccagccctcggggccactcgtggttctgctgcccctggccggtgggtatagccgcatcctcaacgctgcctgccggcacctggcgaggactggggtggtgctggttgcagcagctgggaacttccgggacgacgcctgcctctactccccagcttctgctccagaggtcatcacagtcggggccacgaatgcccaggaccagccagttaccttggggactttggggactaattttggacgctgtgtggatctctttgcccccgggaaggacatcatcggagcgtccagtgactgcagcacatgcttcatgtcacagagtgggacctcacaggctgctgcccacgtggccggcattgtggctcggatgctgagccgggagcccacacttaccctggccgagctgcggcagaggctgatccacttctctaccaaagacgtcatcaacatggcctggttccctgaggaccagcaggtgctgacccccaacctggtggccacactgccccccagcacccatgagacaggcgggcagctgctctgtaggacggtgtggtcggcacactcggggcccactcgaacagctacagctacagcccgctgtgccccagaagaggagctgctgagctgctccagcttctccaggagcgggaggcgtcgtggtgattggattgaggccataggaggccagcaggtctgcaaggccctcaatgcatttgggggtgagggtgtctatgccgtcgcgagatgctgcctggttccccgtgccaactgcagcatccacaacacccctgcagccagagctggcctggagacccatgtccactgccaccagaaggaccatgttctcacaggctgcagcttccattgggaagtggaagaccttagtgtccggaggcagcctgcgctgaggtccagacgtcagcctggccagtgcgttggccaccaggcggccagtgtctatgcttcctgctgccatgccccagggctggaatgcaaaatcaaggagcatgggatctcaggtccttcagagcaggtcactgtggcctgcgaagcaggatggaccctgactggatgcaatgtgctccctggggcatccctcactctgggagcctacagcgtggacaacctgtgtgtggcaagagtccatgacactgccagagcagacaggaccagtggagaagccacagtagctgctgccatctgctgccggagccggccttcagcaaaggcctcctgggttcagtgacagcctcaggcagggatggtgcttgaggctgggtgcagagatatgcctgcatggctctcttgtagccaaaggtggggagattctgcgtgggagaacttggtgtctcaccctgggtacccattcctggtgtatggaagcacctccttcacggtcagggggcctgtgcttggctttctgccatcagacattaagctgtagctggctctggccagctgctccagtgtaccagaacctgaggatgctcgctgcaaggcctcagttctcaggccttagggtgtatttgtctttcaggaagatcataatggacagagatccttggaggttcaaagaccaagtaccagactggaaaattgagtctgaaagccacaaggacagtcaactcacagccagctcacattgcagacaccattttgggctccctgattaaatgcagatcagttctgcacacctccaggggtggatccagctgtaaggccatacctatatcttccagatgtcctcatctgctgcagggctttggccctgctcaggataatgtgctatgagccctcatctgactctcagtttgtactggagaaccatacaggacttaccgcaccttaccccatccactaccatgtgcactgactggcctcattttatgaaggaagagacaggaccagagaggcgatgtcacacagccagtgatgtcaggacataaattcagagtggctggccctgaataatgccaggctgggcagcgagaggacaggctatggcttgctcctggacctatactcccttagccccagtcccacagatcaggtggagactggagtgacagagggcgactgtaccaaggccacaccagctgaccagcacacctctatccttttgagctcttctgtctttttatagtaagcttcctccacctgtgttgcttttgtaacttgatatttatgcagggttttgtagtttttattatgtagtgacttttcagaataaaagcagctgatgtgactgactgcatccg

SEQ ID NO: 17 - DNA, 3731 bp, Homo sapiens
gtccgatggggctctggtggcgtgatctgcgcgccccaggcgtcaagcacccacaccctagaaggtttccgcagcgacgtcgaggcgctcatggttgcaggcgggcgccgccgttcagttcagggtctgagcctggaggagtgagccaggcagtgagactggctcgggcgggccgggacgcgtcgttgcagcagcggctcccagctcccagccaggattccgcgcgccccttcacgcgccctgctcctgaacttcagctcctgcacagtcctccccaccgcaaggctcaaggcgccgccggcgtggaccgcgcacggcctctaggtctcctcgccaggacagcaacctctcccctggccctcatgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccagtgacagccccatcccaggatgggtgtctggggagggtcaagggctggggctgagctttaaaatggttccgacttgtccctctctcagccctccatggcctggcacgaggggatggggatgcttccgcctttccggggctgctggcctggcccttgagtggggcagcctccttgcctggaactcactcactctgggtgcctcctccccaggtggaggtgccaggaagctccctccctcactgtggggcatttcaccattcaaacaggtcgagctgtgctcgggtgctgccagctgctcccaatgtgccgatgtccgtgggcagaatgacttttattgagctcttgttccgtgccaggcattcaatcctcaggtctccaccaaggaggcaggattcttcccatggataggggagggggcggtaggggctgcagggacaaacatcgttggggggtgagtgtgaaaggtgctgatggccctcatctccagctaactgtggagaagcccctgggggctccctgattaatggaggcttagctttctggatggcatctagccagaggctggagacaggtgcgcccctggtggtcacaggctgtgccttggtttcctgagccacctttactctgctctatgccaggctgtgctagcaacacccaaaggtggcctgcggggagccatcacctaggactgactcggcagtgtgcagtggtgcatgcactgtctcagccaacccgctccactacccggcagggtacacattcgcacccctacttcacagaggaagaaacctggaaccagagggggcgtgcctgccaagctcacacagcaggaactgagccagaaacgcagattgggctggctctgaagccaagcctcttcttacttcacccggctgggctcctcatttttacgggtaacagtgaggctgggaaggggaacacagaccaggaagctcggtgagtgatggcagaacgatgcctgcaggcatggaactttttccgttatcacccaggcctgattcactggcctggcggagatgcttctaaggcatggtcgggggagagggccaacaactgtccctccttgagcaccagccccacccaagcaagcagacatttatcttttgggtctgtcctctctgttgcctttttacagccaacttttctagacctgttttgcttttgtaacttgaagatatttattctgggttttgtagcatttttattaatatggtgactttttaaaataaaaacaaacaaacgttgtcctaacaaaaaaaaaaaaaaaaaaaaa